SEQUENCE LISTING (216 sequences)

SEQ ID NO: 1 - RNA, 19 nt, Artificial Sequence
  Description of Artificial Sequence Synthetic oligonucleotide
cccuuccuucgaaaugcaa

SEQ ID NO: 2 - RNA, 19 nt, Artificial Sequence
  Description of Artificial Sequence Synthetic oligonucleotide
ccuuccuucgaaaugcaau

SEQ ID NO: 3 - RNA, 19 nt, Artificial Sequence
  Description of Artificial Sequence Synthetic oligonucleotide
gcugggaagccuucucuaa

SEQ ID NO: 4 - RNA, 19 nt, Artificial Sequence
  Description of Artificial Sequence Synthetic oligonucleotide
ccuccugugccugaugauu

SEQ ID NO: 5 - RNA, 19 nt, Artificial Sequence
  Description of Artificial Sequence Synthetic oligonucleotide
gcagcuuccugauucaaau

SEQ ID NO: 6 - RNA, 19 nt, Artificial Sequence
  Description of Artificial Sequence Synthetic oligonucleotide
gcaacacuugaguggcuau

SEQ ID NO: 7 - RNA, 19 nt, Artificial Sequence
  Description of Artificial Sequence Synthetic oligonucleotide
gcuuuaugcugaagcccua

SEQ ID NO: 8 - RNA, 19 nt, Artificial Sequence
  Description of Artificial Sequence Synthetic oligonucleotide
ccaucuuuggugaaaccau

SEQ ID NO: 9 - RNA, 19 nt, Artificial Sequence
  Description of Artificial Sequence Synthetic oligonucleotide
gcugucccuuuacuucauu

SEQ ID NO: 10 - RNA, 19 nt, Artificial Sequence
  Description of Artificial Sequence Synthetic oligonucleotide
cccaaauuauugguuccaa

SEQ ID NO: 11 - RNA, 25 nt, Artificial Sequence
  Description of Artificial Sequence Synthetic oligonucleotide
gaguuauguguugacauccagauca

SEQ ID NO: 12 - RNA, 25 nt, Artificial Sequence
  Description of Artificial Sequence Synthetic oligonucleotide
cagaucacauuugauugacagucca

SEQ ID NO: 13 - RNA, 25 nt, Artificial Sequence
  Description of Artificial Sequence Synthetic oligonucleotide
gaagccuucucuaaccucuccuauu

SEQ ID NO: 14 - RNA, 25 nt, Artificial Sequence
  Description of Artificial Sequence Synthetic oligonucleotide
cgacucccuugggugucaaagguaa

SEQ ID NO: 15 - RNA, 25 nt, Artificial Sequence
  Description of Artificial Sequence Synthetic oligonucleotide
cagaucauaagcgagggccagcuuu

SEQ ID NO: 16 - RNA, 25 nt, Artificial Sequence
  Description of Artificial Sequence Synthetic oligonucleotide
cagucaaagauacucaggcagagau

SEQ ID NO: 17 - RNA, 25 nt, Artificial Sequence
  Description of Artificial Sequence Synthetic oligonucleotide
uccagacaagcaggcuaauacugau

SEQ ID NO: 18 - RNA, 25 nt, Artificial Sequence
  Description of Artificial Sequence Synthetic oligonucleotide
caacacuugaguggcuaucacuuca

SEQ ID NO: 19 - RNA, 25 nt, Artificial Sequence
  Description of Artificial Sequence Synthetic oligonucleotide
gcaaacgcuuuaugcugaagcccua

SEQ ID NO: 20 - RNA, 25 nt, Artificial Sequence
  Description of Artificial Sequence Synthetic oligonucleotide
acgcuuuaugcugaagcccuaugaa

SEQ ID NO: 21 - RNA, 19 nt, Artificial Sequence
  Description of Artificial Sequence Synthetic oligonucleotide
ccugcgauucaccaacauu

SEQ ID NO: 22 - RNA, 19 nt, Artificial Sequence
  Description of Artificial Sequence Synthetic oligonucleotide
ggucagcaucguugcucuu

SEQ ID NO: 23 - RNA, 19 nt, Artificial Sequence
  Description of Artificial Sequence Synthetic oligonucleotide
gcagagaggaaagucccuu

SEQ ID NO: 24 - RNA, 19 nt, Artificial Sequence
  Description of Artificial Sequence Synthetic oligonucleotide
gcugucacagugagauauu

SEQ ID NO: 25 - RNA, 19 nt, Artificial Sequence
  Description of Artificial Sequence Synthetic oligonucleotide
gcaagucuacagcuaccau

SEQ ID NO: 26 - RNA, 19 nt, Artificial Sequence
  Description of Artificial Sequence Synthetic oligonucleotide
gcaagcagcaagccaauuu

SEQ ID NO: 27 - RNA, 19 nt, Artificial Sequence
  Description of Artificial Sequence Synthetic oligonucleotide
gcagcaagccaauuuccau

SEQ ID NO: 28 - RNA, 19 nt, Artificial Sequence
  Description of Artificial Sequence Synthetic oligonucleotide
gcaagccaauuuccauuaa

SEQ ID NO: 29 - RNA, 19 nt, Artificial Sequence
  Description of Artificial Sequence Synthetic oligonucleotide
ccgaacagaaauugacaaa

SEQ ID NO: 30 - RNA, 19 nt, Artificial Sequence
  Description of Artificial Sequence Synthetic oligonucleotide
gguccagaucaaacagaaa

SEQ ID NO: 31 - RNA, 25 nt, Artificial Sequence
  Description of Artificial Sequence Synthetic oligonucleotide
ccuggugcguuacucaccugugaaa

SEQ ID NO: 32 - RNA, 25 nt, Artificial Sequence
  Description of Artificial Sequence Synthetic oligonucleotide
agucccuuauugauuggccaacaau

SEQ ID NO: 33 - RNA, 25 nt, Artificial Sequence
  Description of Artificial Sequence Synthetic oligonucleotide
cagugagauauuacaggaucacuua

SEQ ID NO: 34 - RNA, 25 nt, Artificial Sequence
  Description of Artificial Sequence Synthetic oligonucleotide
cagcggccuuaaaccuggaguugau

SEQ ID NO: 35 - RNA, 25 nt, Artificial Sequence
  Description of Artificial Sequence Synthetic oligonucleotide
gaguugauuauaccaucacugugua

SEQ ID NO: 36 - RNA, 25 nt, Artificial Sequence
  Description of Artificial Sequence Synthetic oligonucleotide
ccgcaagcagcaagccaauuuccau

SEQ ID NO: 37 - RNA, 25 nt, Artificial Sequence
  Description of Artificial Sequence Synthetic oligonucleotide
aagcagcaagccaauuuccauuaau

SEQ ID NO: 38 - RNA, 25 nt, Artificial Sequence
  Description of Artificial Sequence Synthetic oligonucleotide
agcagcaagccaauuuccauuaauu

SEQ ID NO: 39 - RNA, 25 nt, Artificial Sequence
  Description of Artificial Sequence Synthetic oligonucleotide
cagcccacaguggaguaugugguua

SEQ ID NO: 40 - RNA, 25 nt, Artificial Sequence
  Description of Artificial Sequence Synthetic oligonucleotide
caguggaguaugugguuagugucua

SEQ ID NO: 41 - RNA, 19 nt, Artificial Sequence
  Description of Artificial Sequence Synthetic oligonucleotide
ccggcaauuaugccaccuu

SEQ ID NO: 42 - RNA, 19 nt, Artificial Sequence
  Description of Artificial Sequence Synthetic oligonucleotide
ccaaggauaucgaaggcuu

SEQ ID NO: 43 - RNA, 19 nt, Artificial Sequence
  Description of Artificial Sequence Synthetic oligonucleotide
ccaaagcaaugccacccau

SEQ ID NO: 44 - RNA, 19 nt, Artificial Sequence
  Description of Artificial Sequence Synthetic oligonucleotide
ccgugccuuaugguuacuu

SEQ ID NO: 45 - RNA, 19 nt, Artificial Sequence
  Description of Artificial Sequence Synthetic oligonucleotide
gggaguaugcggcuaacaa

SEQ ID NO: 46 - RNA, 19 nt, Artificial Sequence
  Description of Artificial Sequence Synthetic oligonucleotide
ggucaaagagaagaagguu

SEQ ID NO: 47 - RNA, 19 nt, Artificial Sequence
  Description of Artificial Sequence Synthetic oligonucleotide
cccagucauaaucauucau

SEQ ID NO: 48 - RNA, 19 nt, Artificial Sequence
  Description of Artificial Sequence Synthetic oligonucleotide
ccaugaucguuagccucau

SEQ ID NO: 49 - RNA, 19 nt, Artificial Sequence
  Description of Artificial Sequence Synthetic oligonucleotide
gcacuuuagaaaccgcuuu

SEQ ID NO: 50 - RNA, 19 nt, Artificial Sequence
  Description of Artificial Sequence Synthetic oligonucleotide
gcuuucaugaauugagcua

SEQ ID NO: 51 - RNA, 25 nt, Artificial Sequence
  Description of Artificial Sequence Synthetic oligonucleotide
gguuacuuuggaggcggguacuacu

SEQ ID NO: 52 - RNA, 25 nt, Artificial Sequence
  Description of Artificial Sequence Synthetic oligonucleotide
cgggaguaugcggcuaacaaguuca

SEQ ID NO: 53 - RNA, 25 nt, Artificial Sequence
  Description of Artificial Sequence Synthetic oligonucleotide
gaguaugcggcuaacaaguucauca

SEQ ID NO: 54 - RNA, 25 nt, Artificial Sequence
  Description of Artificial Sequence Synthetic oligonucleotide
gagaagaagguucucgccaagguga

SEQ ID NO: 55 - RNA, 25 nt, Artificial Sequence
  Description of Artificial Sequence Synthetic oligonucleotide
cccaaagaaccuggcccagucauaa

SEQ ID NO: 56 - RNA, 25 nt, Artificial Sequence
  Description of Artificial Sequence Synthetic oligonucleotide
cagucauaaucauucauccugacag

SEQ ID NO: 57 - RNA, 25 nt, Artificial Sequence
  Description of Artificial Sequence Synthetic oligonucleotide
cauucauccugacaguggcaauaau

SEQ ID NO: 58 - RNA, 25 nt, Artificial Sequence
  Description of Artificial Sequence Synthetic oligonucleotide
uagagcucuguagagcacuuuagaa

SEQ ID NO: 59 - RNA, 25 nt, Artificial Sequence
  Description of Artificial Sequence Synthetic oligonucleotide
gagcacuuuagaaaccgcuuucaug

SEQ ID NO: 60 - RNA, 25 nt, Artificial Sequence
  Description of Artificial Sequence Synthetic oligonucleotide
ccgcuuucaugaauugagcuaauua

SEQ ID NO: 61 - RNA, 19 nt, Artificial Sequence
  Description of Artificial Sequence Synthetic oligonucleotide
gcaucucagcccugagaaa

SEQ ID NO: 62 - RNA, 19 nt, Artificial Sequence
  Description of Artificial Sequence Synthetic oligonucleotide
gcccugagaaaggagacau

SEQ ID NO: 63 - RNA, 19 nt, Artificial Sequence
  Description of Artificial Sequence Synthetic oligonucleotide
ggaugcuuccaaucuggau

SEQ ID NO: 64 - RNA, 19 nt, Artificial Sequence
  Description of Artificial Sequence Synthetic oligonucleotide
gcuuccaaucuggauucaa

SEQ ID NO: 65 - RNA, 19 nt, Artificial Sequence
  Description of Artificial Sequence Synthetic oligonucleotide
ggauucaaugaggagacuu

SEQ ID NO: 66 - RNA, 19 nt, Artificial Sequence
  Description of Artificial Sequence Synthetic oligonucleotide
gcaggacaugacaacucau

SEQ ID NO: 67 - RNA, 19 nt, Artificial Sequence
  Description of Artificial Sequence Synthetic oligonucleotide
ggcaccucagauuguuguu

SEQ ID NO: 68 - RNA, 19 nt, Artificial Sequence
  Description of Artificial Sequence Synthetic oligonucleotide
gcacagaacuuauguuguu

SEQ ID NO: 69 - RNA, 19 nt, Artificial Sequence
  Description of Artificial Sequence Synthetic oligonucleotide
ggaaaguggcuaugcaguu

SEQ ID NO: 70 - RNA, 19 nt, Artificial Sequence
  Description of Artificial Sequence Synthetic oligonucleotide
gaaaguggcuaugcaguuu

SEQ ID NO: 71 - RNA, 25 nt, Artificial Sequence
  Description of Artificial Sequence Synthetic oligonucleotide
cagcccugagaaaggagacauguaa

SEQ ID NO: 72 - RNA, 25 nt, Artificial Sequence
  Description of Artificial Sequence Synthetic oligonucleotide
uggaugcuuccaaucuggauucaau

SEQ ID NO: 73 - RNA, 25 nt, Artificial Sequence
  Description of Artificial Sequence Synthetic oligonucleotide
gagguauaccuagaguaccuccaga

SEQ ID NO: 74 - RNA, 25 nt, Artificial Sequence
  Description of Artificial Sequence Synthetic oligonucleotide
ccuccagaacagauuugagaguagu

SEQ ID NO: 75 - RNA, 25 nt, Artificial Sequence
  Description of Artificial Sequence Synthetic oligonucleotide
caacucaucucauucugcgcagcuu

SEQ ID NO: 76 - RNA, 25 nt, Artificial Sequence
  Description of Artificial Sequence Synthetic oligonucleotide
gggcaccucagauuguuguuguuaa

SEQ ID NO: 77 - RNA, 25 nt, Artificial Sequence
  Description of Artificial Sequence Synthetic oligonucleotide
cacugggcacagaacuuauguuguu

SEQ ID NO: 78 - RNA, 25 nt, Artificial Sequence
  Description of Artificial Sequence Synthetic oligonucleotide
ggcacagaacuuauguuguucucua

SEQ ID NO: 79 - RNA, 25 nt, Artificial Sequence
  Description of Artificial Sequence Synthetic oligonucleotide
gcacagaacuuauguuguucucuau

SEQ ID NO: 80 - RNA, 25 nt, Artificial Sequence
  Description of Artificial Sequence Synthetic oligonucleotide
uggaaaguguaggcuuaccucaaau

SEQ ID NO: 81 - RNA, 19 nt, Artificial Sequence
  Description of Artificial Sequence Synthetic oligonucleotide
uacucccagucuugucauu

SEQ ID NO: 82 - RNA, 19 nt, Artificial Sequence
  Description of Artificial Sequence Synthetic oligonucleotide
acucccagucuugucauug

SEQ ID NO: 83 - RNA, 19 nt, Artificial Sequence
  Description of Artificial Sequence Synthetic oligonucleotide
cccagucuugucauugcca

SEQ ID NO: 84 - RNA, 19 nt, Artificial Sequence
  Description of Artificial Sequence Synthetic oligonucleotide
ccagucuugucauugccag

SEQ ID NO: 85 - RNA, 19 nt, Artificial Sequence
  Description of Artificial Sequence Synthetic oligonucleotide
gcuguguugguagugcugu

SEQ ID NO: 86 - RNA, 19 nt, Artificial Sequence
  Description of Artificial Sequence Synthetic oligonucleotide
ugguagugcuguguugaau

SEQ ID NO: 87 - RNA, 19 nt, Artificial Sequence
  Description of Artificial Sequence Synthetic oligonucleotide
gguagugcuguguugaauu

SEQ ID NO: 88 - RNA, 19 nt, Artificial Sequence
  Description of Artificial Sequence Synthetic oligonucleotide
guagugcuguguugaauua

SEQ ID NO: 89 - RNA, 19 nt, Artificial Sequence
  Description of Artificial Sequence Synthetic oligonucleotide
gcuguguugaauuacggaa

SEQ ID NO: 90 - RNA, 19 nt, Artificial Sequence
  Description of Artificial Sequence Synthetic oligonucleotide
cuguguugaauuacggaau

SEQ ID NO: 91 - RNA, 25 nt, Artificial Sequence
  Description of Artificial Sequence Synthetic oligonucleotide
gcuguguugguagugcuguguugaa

SEQ ID NO: 92 - RNA, 25 nt, Artificial Sequence
  Description of Artificial Sequence Synthetic oligonucleotide
uguguugguagugcuguguugaauu

SEQ ID NO: 93 - RNA, 25 nt, Artificial Sequence
  Description of Artificial Sequence Synthetic oligonucleotide
gguagugcuguguugaauuacggaa

SEQ ID NO: 94 - RNA, 25 nt, Artificial Sequence
  Description of Artificial Sequence Synthetic oligonucleotide
uagugcuguguugaauuacggaaua

SEQ ID NO: 95 - RNA, 25 nt, Artificial Sequence
  Description of Artificial Sequence Synthetic oligonucleotide
agugcuguguugaauuacggaauaa

SEQ ID NO: 96 - RNA, 25 nt, Artificial Sequence
  Description of Artificial Sequence Synthetic oligonucleotide
ugcuguguugaauuacggaauaaug

SEQ ID NO: 97 - RNA, 25 nt, Artificial Sequence
  Description of Artificial Sequence Synthetic oligonucleotide
gcuguguugaauuacggaauaauga

SEQ ID NO: 98 - RNA, 19 nt, Artificial Sequence
  Description of Artificial Sequence Synthetic oligonucleotide
ggaaagcgggaagacucau

SEQ ID NO: 99 - RNA, 19 nt, Artificial Sequence
  Description of Artificial Sequence Synthetic oligonucleotide
ccuguccauuggacuguaa

SEQ ID NO: 100 - RNA, 19 nt, Artificial Sequence
  Description of Artificial Sequence Synthetic oligonucleotide
ccauuggacuguaagguuu

SEQ ID NO: 101 - RNA, 19 nt, Artificial Sequence
  Description of Artificial Sequence Synthetic oligonucleotide
gcugucucgggagagaaau

SEQ ID NO: 102 - RNA, 19 nt, Artificial Sequence
  Description of Artificial Sequence Synthetic oligonucleotide
gguguacaggaaauuacuu

SEQ ID NO: 103 - RNA, 19 nt, Artificial Sequence
  Description of Artificial Sequence Synthetic oligonucleotide
gguguaauucucuaugguu

SEQ ID NO: 104 - RNA, 19 nt, Artificial Sequence
  Description of Artificial Sequence Synthetic oligonucleotide
ggcauguaauaccaagaau

SEQ ID NO: 105 - RNA, 19 nt, Artificial Sequence
  Description of Artificial Sequence Synthetic oligonucleotide
ccuauuggaagccauacuu

SEQ ID NO: 106 - RNA, 19 nt, Artificial Sequence
  Description of Artificial Sequence Synthetic oligonucleotide
ggcacuauggauuagucuu

SEQ ID NO: 107 - RNA, 19 nt, Artificial Sequence
  Description of Artificial Sequence Synthetic oligonucleotide
gcagguguuguaauuucaa

SEQ ID NO: 108 - RNA, 25 nt, Artificial Sequence
  Description of Artificial Sequence Synthetic oligonucleotide
cccuagaucucgaaaugcaucguga

SEQ ID NO: 109 - RNA, 25 nt, Artificial Sequence
  Description of Artificial Sequence Synthetic oligonucleotide
uccuguccauuggacuguaagguuu

SEQ ID NO: 110 - RNA, 25 nt, Artificial Sequence
  Description of Artificial Sequence Synthetic oligonucleotide
ccuguccauuggacuguaagguuua

SEQ ID NO: 111 - RNA, 25 nt, Artificial Sequence
  Description of Artificial Sequence Synthetic oligonucleotide
cguagucgaucuaggucaaaugaaa

SEQ ID NO: 112 - RNA, 25 nt, Artificial Sequence
  Description of Artificial Sequence Synthetic oligonucleotide
gcaagagaagugguguacaggaaau

SEQ ID NO: 113 - RNA, 25 nt, Artificial Sequence
  Description of Artificial Sequence Synthetic oligonucleotide
cacaaagguguaauucucuaugguu

SEQ ID NO: 114 - RNA, 25 nt, Artificial Sequence
  Description of Artificial Sequence Synthetic oligonucleotide
gagcuugguaccaaguccagguaua

SEQ ID NO: 115 - RNA, 25 nt, Artificial Sequence
  Description of Artificial Sequence Synthetic oligonucleotide
cauuccuauuggaagccauacuuau

SEQ ID NO: 116 - RNA, 25 nt, Artificial Sequence
  Description of Artificial Sequence Synthetic oligonucleotide
aagcaguugguuacacgauucuuau

SEQ ID NO: 117 - RNA, 25 nt, Artificial Sequence
  Description of Artificial Sequence Synthetic oligonucleotide
aggcacuauggauuagucuucugaa

SEQ ID NO: 118 - RNA, 19 nt, Artificial Sequence
  Description of Artificial Sequence Synthetic oligonucleotide
ccagaaauacagcaacaau

SEQ ID NO: 119 - RNA, 19 nt, Artificial Sequence
  Description of Artificial Sequence Synthetic oligonucleotide
gcaacaauuccuggcgaua

SEQ ID NO: 120 - RNA, 19 nt, Artificial Sequence
  Description of Artificial Sequence Synthetic oligonucleotide
ccugugacagcagggauaa

SEQ ID NO: 121 - RNA, 19 nt, Artificial Sequence
  Description of Artificial Sequence Synthetic oligonucleotide
ggacaucaacggguucacu

SEQ ID NO: 122 - RNA, 19 nt, Artificial Sequence
  Description of Artificial Sequence Synthetic oligonucleotide
ccaccauucauggcaugaa

SEQ ID NO: 123 - RNA, 19 nt, Artificial Sequence
  Description of Artificial Sequence Synthetic oligonucleotide
ggccuuuccugcuucucau

SEQ ID NO: 124 - RNA, 19 nt, Artificial Sequence
  Description of Artificial Sequence Synthetic oligonucleotide
gcccuggacaccaacuauu

SEQ ID NO: 125 - RNA, 19 nt, Artificial Sequence
  Description of Artificial Sequence Synthetic oligonucleotide
ggcagcuguacauugacuu

SEQ ID NO: 126 - RNA, 19 nt, Artificial Sequence
  Description of Artificial Sequence Synthetic oligonucleotide
gcccuguacaaccagcaua

SEQ ID NO: 127 - RNA, 19 nt, Artificial Sequence
  Description of Artificial Sequence Synthetic oligonucleotide
cccuguacaaccagcauaa

SEQ ID NO: 128 - RNA, 25 nt, Artificial Sequence
  Description of Artificial Sequence Synthetic oligonucleotide
cagcacguggagcuguaccagaaau

SEQ ID NO: 129 - RNA, 25 nt, Artificial Sequence
  Description of Artificial Sequence Synthetic oligonucleotide
cacguggagcuguaccagaaauaca

SEQ ID NO: 130 - RNA, 25 nt, Artificial Sequence
  Description of Artificial Sequence Synthetic oligonucleotide
gagcuguaccagaaauacagcaaca

SEQ ID NO: 131 - RNA, 25 nt, Artificial Sequence
  Description of Artificial Sequence Synthetic oligonucleotide
agcgacucgccagagugguuaucuu

SEQ ID NO: 132 - RNA, 25 nt, Artificial Sequence
  Description of Artificial Sequence Synthetic oligonucleotide
gcgacucgccagagugguuaucuuu

SEQ ID NO: 133 - RNA, 25 nt, Artificial Sequence
  Description of Artificial Sequence Synthetic oligonucleotide
gcagggauaacacacugcaagugga

SEQ ID NO: 134 - RNA, 25 nt, Artificial Sequence
  Description of Artificial Sequence Synthetic oligonucleotide
acacugcaaguggacaucaacgggu

SEQ ID NO: 135 - RNA, 25 nt, Artificial Sequence
  Description of Artificial Sequence Synthetic oligonucleotide
caaguggacaucaacggguucacua

SEQ ID NO: 136 - RNA, 25 nt, Artificial Sequence
  Description of Artificial Sequence Synthetic oligonucleotide
ugaaccggccuuuccugcuucucau

SEQ ID NO: 137 - RNA, 25 nt, Artificial Sequence
  Description of Artificial Sequence Synthetic oligonucleotide
gacaccaacuauugcuucagcucca

SEQ ID NO: 138 - RNA, 19 nt, Artificial Sequence
  Description of Artificial Sequence Synthetic oligonucleotide
ccugcagcacacucgauau

SEQ ID NO: 139 - RNA, 19 nt, Artificial Sequence
  Description of Artificial Sequence Synthetic oligonucleotide
gcgcuacaucgacagcaaa

SEQ ID NO: 140 - RNA, 19 nt, Artificial Sequence
  Description of Artificial Sequence Synthetic oligonucleotide
gcuuuggaugcggccuauu

SEQ ID NO: 141 - RNA, 19 nt, Artificial Sequence
  Description of Artificial Sequence Synthetic oligonucleotide
ggaugcggccuauugcuuu

SEQ ID NO: 142 - RNA, 19 nt, Artificial Sequence
  Description of Artificial Sequence Synthetic oligonucleotide
gcugccuacguccacuuua

SEQ ID NO: 143 - RNA, 19 nt, Artificial Sequence
  Description of Artificial Sequence Synthetic oligonucleotide
gccuacguccacuuuacau

SEQ ID NO: 144 - RNA, 19 nt, Artificial Sequence
  Description of Artificial Sequence Synthetic oligonucleotide
ccuacguccacuuuacauu

SEQ ID NO: 145 - RNA, 19 nt, Artificial Sequence
  Description of Artificial Sequence Synthetic oligonucleotide
ccaaaggguacaaugccaa

SEQ ID NO: 146 - RNA, 19 nt, Artificial Sequence
  Description of Artificial Sequence Synthetic oligonucleotide
gguccugagcuuauauaau

SEQ ID NO: 147 - RNA, 19 nt, Artificial Sequence
  Description of Artificial Sequence Synthetic oligonucleotide
gcugcgugucccaagauuu

SEQ ID NO: 148 - RNA, 25 nt, Artificial Sequence
  Description of Artificial Sequence Synthetic oligonucleotide
caaguccaaagauuuaacaucucca

SEQ ID NO: 149 - RNA, 25 nt, Artificial Sequence
  Description of Artificial Sequence Synthetic oligonucleotide
cgauguaacugaugcuguucaugaa

SEQ ID NO: 150 - RNA, 25 nt, Artificial Sequence
  Description of Artificial Sequence Synthetic oligonucleotide
acuagaagcaagauuugcagguauu

SEQ ID NO: 151 - RNA, 25 nt, Artificial Sequence
  Description of Artificial Sequence Synthetic oligonucleotide
gagggaucuaggguggaaauggaua

SEQ ID NO: 152 - RNA, 25 nt, Artificial Sequence
  Description of Artificial Sequence Synthetic oligonucleotide
cccaaaggguacaaugccaacuucu

SEQ ID NO: 153 - RNA, 25 nt, Artificial Sequence
  Description of Artificial Sequence Synthetic oligonucleotide
caaugccaacuucugugcuggagca

SEQ ID NO: 154 - RNA, 25 nt, Artificial Sequence
  Description of Artificial Sequence Synthetic oligonucleotide
gguccugagcuuauauaauaccaua

SEQ ID NO: 155 - RNA, 25 nt, Artificial Sequence
  Description of Artificial Sequence Synthetic oligonucleotide
cgugucccaagauuuagaaccucua

SEQ ID NO: 156 - RNA, 25 nt, Artificial Sequence
  Description of Artificial Sequence Synthetic oligonucleotide
ccaagauuuagaaccucuaaccauu

SEQ ID NO: 157 - RNA, 25 nt, Artificial Sequence
  Description of Artificial Sequence Synthetic oligonucleotide
cacccaagauugaacagcuuucuaa

SEQ ID NO: 158 - RNA, 19 nt, Artificial Sequence
  Description of Artificial Sequence Synthetic oligonucleotide
gccuggucaagaaacucaa

SEQ ID NO: 159 - RNA, 19 nt, Artificial Sequence
  Description of Artificial Sequence Synthetic oligonucleotide
gcgugaaucccuaccacua

SEQ ID NO: 160 - RNA, 19 nt, Artificial Sequence
  Description of Artificial Sequence Synthetic oligonucleotide
gccauccaugacuguggau

SEQ ID NO: 161 - RNA, 19 nt, Artificial Sequence
  Description of Artificial Sequence Synthetic oligonucleotide
ccaugacuguggauggcuu

SEQ ID NO: 162 - RNA, 19 nt, Artificial Sequence
  Description of Artificial Sequence Synthetic oligonucleotide
gcaaccugaagaucuucaa

SEQ ID NO: 163 - RNA, 19 nt, Artificial Sequence
  Description of Artificial Sequence Synthetic oligonucleotide
ccgcaugagcuucgucaaa

SEQ ID NO: 164 - RNA, 19 nt, Artificial Sequence
  Description of Artificial Sequence Synthetic oligonucleotide
ggauugagcugcaccugaa

SEQ ID NO: 165 - RNA, 19 nt, Artificial Sequence
  Description of Artificial Sequence Synthetic oligonucleotide
gcuguuccagugugucuua

SEQ ID NO: 166 - RNA, 19 nt, Artificial Sequence
  Description of Artificial Sequence Synthetic oligonucleotide
ggaacucuacucaacccau

SEQ ID NO: 167 - RNA, 19 nt, Artificial Sequence
  Description of Artificial Sequence Synthetic oligonucleotide
ccaaacacauuuacccuuu

SEQ ID NO: 168 - RNA, 25 nt, Artificial Sequence
  Description of Artificial Sequence Synthetic oligonucleotide
ccagagaguagagacaccaguucua

SEQ ID NO: 169 - RNA, 25 nt, Artificial Sequence
  Description of Artificial Sequence Synthetic oligonucleotide
gagaaaccagugaccaccagaugaa

SEQ ID NO: 170 - RNA, 25 nt, Artificial Sequence
  Description of Artificial Sequence Synthetic oligonucleotide
cagcacauaauaacuuggaccugca

SEQ ID NO: 171 - RNA, 25 nt, Artificial Sequence
  Description of Artificial Sequence Synthetic oligonucleotide
caccaggaugcaaccugaagaucuu

SEQ ID NO: 172 - RNA, 25 nt, Artificial Sequence
  Description of Artificial Sequence Synthetic oligonucleotide
ccagugugucuuagagacaucaagu

SEQ ID NO: 173 - RNA, 25 nt, Artificial Sequence
  Description of Artificial Sequence Synthetic oligonucleotide
cagugugucuuagagacaucaagua

SEQ ID NO: 174 - RNA, 25 nt, Artificial Sequence
  Description of Artificial Sequence Synthetic oligonucleotide
aagaaaucuuucucccucaacugaa

SEQ ID NO: 175 - RNA, 25 nt, Artificial Sequence
  Description of Artificial Sequence Synthetic oligonucleotide
cgagcaaacccagagguggauguua

SEQ ID NO: 176 - RNA, 25 nt, Artificial Sequence
  Description of Artificial Sequence Synthetic oligonucleotide
gagcaaacccagagguggauguuau

SEQ ID NO: 177 - RNA, 25 nt, Artificial Sequence
  Description of Artificial Sequence Synthetic oligonucleotide
gagguggauguuaugaacagcugug

SEQ ID NO: 178 - DNA, 22 bp, Artificial Sequence
  Description of Artificial Sequence Synthetic primer
cggagctctgatgtgttgaaga

SEQ ID NO: 179 - DNA, 22 bp, Artificial Sequence
  Description of Artificial Sequence Synthetic primer
ggctgcggctgctgccgctgct

SEQ ID NO: 180 - DNA, 22 bp, Artificial Sequence
  Description of Artificial Sequence Synthetic primer
gcgtagtagtcggcctcaggct

SEQ ID NO: 181 - DNA, 22 bp, Artificial Sequence
  Description of Artificial Sequence Synthetic primer
gcagcagggacagtgtaagctt

SEQ ID NO: 182 - DNA, 22 bp, Artificial Sequence
  Description of Artificial Sequence Synthetic primer
gccgcctgcgagcgcgagagga

SEQ ID NO: 183 - DNA, 22 bp, Artificial Sequence
  Description of Artificial Sequence Synthetic primer
gctgtcgatgtagcgctgggtt

SEQ ID NO: 184 - DNA, 22 bp, Artificial Sequence
  Description of Artificial Sequence Synthetic primer
ctcctgtttaagcacatcgcat

SEQ ID NO: 185 - DNA, 22 bp, Artificial Sequence
  Description of Artificial Sequence Synthetic primer
gcttcctgattcaaatgagatt

SEQ ID NO: 186 - DNA, 22 bp, Artificial Sequence
  Description of Artificial Sequence Synthetic primer
ctctccatcaattatctgatat

SEQ ID NO: 187 - DNA, 22 bp, Artificial Sequence
  Description of Artificial Sequence Synthetic primer
gctgtcacatggggttccgtct

SEQ ID NO: 188 - DNA, 22 bp, Artificial Sequence
  Description of Artificial Sequence Synthetic primer
gggcagcaccctcctgacgcct

SEQ ID NO: 189 - DNA, 22 bp, Artificial Sequence
  Description of Artificial Sequence Synthetic primer
cccagcctgggcttggcaggtt

SEQ ID NO: 190 - DNA, 22 bp, Artificial Sequence
  Description of Artificial Sequence Synthetic primer
gtggttactctgtaaccagtaa

SEQ ID NO: 191 - DNA, 22 bp, Artificial Sequence
  Description of Artificial Sequence Synthetic primer
ctccaggcacagagtatgtggt

SEQ ID NO: 192 - DNA, 22 bp, Artificial Sequence
  Description of Artificial Sequence Synthetic primer
cagtgacagcatacacagtgat

SEQ ID NO: 193 - DNA, 22 bp, Artificial Sequence
  Description of Artificial Sequence Synthetic primer
gcagcatttcgttttccctgat

SEQ ID NO: 194 - DNA, 22 bp, Artificial Sequence
  Description of Artificial Sequence Synthetic primer
ggtcaaatgaaaggaaatagaa

SEQ ID NO: 195 - DNA, 22 bp, Artificial Sequence
  Description of Artificial Sequence Synthetic primer
ggtttattatcagtctgtgcat

SEQ ID NO: 196 - DNA, 22 bp, Artificial Sequence
  Description of Artificial Sequence Synthetic primer
ctgcatagccactttccattat

SEQ ID NO: 197 - DNA, 22 bp, Artificial Sequence
  Description of Artificial Sequence Synthetic primer
gcagcaaagaggcactggcaga

SEQ ID NO: 198 - DNA, 22 bp, Artificial Sequence
  Description of Artificial Sequence Synthetic primer
cagcttcgtcagcaggctggca

SEQ ID NO: 199 - DNA, 22 bp, Artificial Sequence
  Description of Artificial Sequence Synthetic primer
gggttgccagatttaacagaaa

SEQ ID NO: 200 - DNA, 22 bp, Artificial Sequence
  Description of Artificial Sequence Synthetic primer
gaatcagtgaagatgccagtga

SEQ ID NO: 201 - DNA, 20 bp, Artificial Sequence
  Description of Artificial Sequence Synthetic primer
cctgaaattaaagttcggat

SEQ ID NO: 202 - DNA, 22 bp, Artificial Sequence
  Description of Artificial Sequence Synthetic primer
ctgcattcctgttgacattgga

SEQ ID NO: 203 - DNA, 22 bp, Artificial Sequence
  Description of Artificial Sequence Synthetic primer
gggctccctcatgtcatctact

SEQ ID NO: 204 - DNA, 22 bp, Artificial Sequence
  Description of Artificial Sequence Synthetic primer
cgtagtaggagatggagcacca

SEQ ID NO: 205 - RNA, 19 nt, Artificial Sequence
  Description of Artificial Sequence Synthetic oligonucleotide
cccuuccuucgaaaugcaa

SEQ ID NO: 206 - RNA, 19 nt, Artificial Sequence
  Description of Artificial Sequence Synthetic oligonucleotide
ccagaaauacagcaacaau

SEQ ID NO: 207 - RNA, 19 nt, Artificial Sequence
  Description of Artificial Sequence Synthetic oligonucleotide
ccugcagcacacucgauau

SEQ ID NO: 208 - RNA, 19 nt, Artificial Sequence
  Description of Artificial Sequence Synthetic oligonucleotide
cccuuccuucgaaaugcaa

SEQ ID NO: 209 - RNA, 19 nt, Artificial Sequence
  Description of Artificial Sequence Synthetic oligonucleotide
ccagaaauacagcaacaau

SEQ ID NO: 210 - RNA, 19 nt, Artificial Sequence
  Description of Artificial Sequence Synthetic oligonucleotide
ccggcaauuaugccaccuu

SEQ ID NO: 211 - RNA, 19 nt, Artificial Sequence
  Description of Artificial Sequence Synthetic oligonucleotide
ggaaagcgggaagacucau

SEQ ID NO: 212 - RNA, 19 nt, Artificial Sequence
  Description of Artificial Sequence Synthetic oligonucleotide
ccagaaauacagcaacaau

SEQ ID NO: 213 - RNA, 19 nt, Artificial Sequence
  Description of Artificial Sequence Synthetic oligonucleotide
ccggcaauuaugccaccuu

SEQ ID NO: 214 - RNA, 19 nt, Artificial Sequence
  Description of Artificial Sequence Synthetic oligonucleotide
gcaucucagcccugagaaa

SEQ ID NO: 215 - RNA, 19 nt, Artificial Sequence
  Description of Artificial Sequence Synthetic oligonucleotide
ccagaaauacagcaacaau

SEQ ID NO: 216 - RNA, 19 nt, Artificial Sequence
  Description of Artificial Sequence Synthetic oligonucleotide
ccggcaauuaugccaccuu

(19) United States
(12) Patent Application Publication
(10) Pub. No.: US 2012/0115923 A1
(43) Pub. Date: May 10, 2012

(54) Sirna Compositions Promoting Scar-Free Wound Healing of Skin and Methods for Wound Treatment

(75) Inventors: Wei-Wu He, Potomac, MD (US); Patrick Y. Lu, Rockville, MD (US)

(73) Assignee: Intradigm Corporation, Pal Alto, CA (US)

(21) Appl. No.: 12/087,413

(22) PCT Filed: Dec. 29, 2006
(86) PCT No.: PCT/US2006/049575

§ 371 Date: Mar. 8, 2010

Related U.S. Application Data
(60) Provisional application No. 60/755,549, filed on Dec. 30, 2005.

(51) Int. Cl. A61K 31/713; C12N 15/113; C12N 5/071; A61P 29/00; A61P 17/02; C12N 15/11; A61K 31/7105

(52) U.S. Cl. 514/44 A; 536/23.1; 536/24.5; 514/44 R; 435/375

(57) ABSTRACT

This invention describes compositions and methods using siRNA to target various genes expressed in cells of injured tissue during scar formation to promote scar-free wound healing.

This application claims the benefit of U.S. provisional application No. 60/755,549, the entire disclosure of which is incorporated herein by reference.

### FIELD OF THE INVENTION

The present invention relates to concepts, compositions and methods for prevention and minimization of skin scar formation during the wound healing process, using siRNA agents to knockdown expressions of genes promoting skin scar formation. The siRNA agent can be used as either single duplex or multiple duplexes (cocktail), targeting either single or multiple genes, with or without transfection carriers. The transfection agents include but not limited to synthetic polymers, liposome and sugars, etc., when they are applied with other skin care materials. The siRNA agents can also be used with other agents such as small molecule and monoclonal antibody inhibitors, immune modulators and other types of oligos, in the same application. The injection, topic and transdermal administrations of siRNA agents are all applicable for the wound healing process following cutaneous tissue injury and skin grafts. The present invention is a novel treatment to enhance skin scarless healing from wounds, caused by burns, chronic skin ulcers, general surgery, plastic surgery and accidental cuts, etc. The invention is useful for pharmaceutical and cosmaceutical industries.

### BACKGROUND OF THE INVENTION

The primary function of the skin is to serve as a protective barrier against the environment. Loss of the integrity of large portions of the skin as a result of injury or illness may lead to major disability or even death. Every year in the United States more than 1.25 million people have burns and 6.5 million have chronic skin ulcers caused by pressure, venous stasis, or diabetes mellitus. The primary goals of the treatment of wounds are rapid wound closure and a functional and aesthetically satisfactory scar. Recent advances in cellular and molecular biology have greatly expanded our understanding of the biologic processes involved in wound repair and tissue regeneration and have led to improvements in wound care. Wound healing is a dynamic, interactive process involving soluble mediators, blood cells, extracellular matrix, and parenchymal cells. Wound healing has three phases—inflammation, tissue formation, and tissue remodeling—that overlap in time (1-3).

The cutaneous wound healing process is known to differ between fetal and adult skin. Wound repair in adult skin begins with an acute inflammatory phase and ends with the formation of a permanent scar. In contrast, early gestation fetal wounds (first and second trimester) heal in a near perfect fashion, rapidly and without the production of a scar. There has been much interest in characterizing the key factors responsible for the switch from scarless healing to an adult-like, scar-producing phenotype typical of skin past the second trimester of gestation. Identification of differences in the two types of healing could identify factors that promote scar tissue generation. This correlation between factors identified as reduced in scarless healing and the inhibition of those factors in adult wounds to reduce scarring has been especially true for transforming growth factor-Bβ (TGF-β). This cytokine was one of the first mediators found to be differentially regulated in scarless healing and was shown to promote scar tissue deposition when introduced into scarless wounds (4-7). As a result of these findings and others implicating TGF-β in fibrosis, the effect of down-regulating this molecule was tested in adult skin and found to reduce scar formation (8-15).

The fetal response to cutaneous injury differs markedly from that of the adult, proceeding with only minimal inflammation, minimal fibroblast proliferation, and only essential collagen deposition. The effect of platelet-derived growth factor (PDGF) on both cellular and extracellular matrix events at a fetal wound site has been investigated because PDGF is known to play an important role in adult wound healing regulation. SILASTIC wound implants were harvested after either 1, 3, or 5 days in utero. The specimens underwent standard histological processing and were evaluated. PDGF-treated implants had a marked increase in acute inflammation, fibroblast recruitment, and collagen and hyaluronic acid deposition. These differences appeared to be largely time- and PDGF dose-dependent and the data suggest that fetal repair proceeds in the absence of PDGF (16).

A key feature of scarless fetal healing appears to be a lack of inflammation in response to the wounding event. In contrast, the early phases of wound healing in late fetal and adult skin are characterized by a robust inflammatory response, and eventually a permanent scar in the wound area. While the interleukins IL-6 (17) and IL-8 (18) have been studied in fetal wound repair, the role of other classic inflammatory mediators in scarless healing is not known.

Metabolites and enzymes of the arachidonic acid cascade, including the cyclooxygenase-2 (COX-2) enzyme and its enzymatic product prostaglandin E2 (PGE2), are known to be critical mediators of the inflammatory response (19-22). COX-2 has received much attention recently as it is involved in diseases associated with dysregulated inflammatory conditions, such as rheumatoid and osteoarthritis, cardiovascular disease, and the carcinogenesis process. COX-2 undergoes immediate-early up-regulation in response to an inflammatory stimulus, such as a wound. It functions by producing prostaglandins that control many aspects of the resulting inflammation, including the induction of vascular permeability and the infiltration and activation of inflammatory cells. Interest in the role of the COX-2 pathway and other aspects of inflammation in the adult wound repair process is increasing as these early events have been shown to regulate the outcome of repair. Based on the involvement of COX-2 in inflammation and the recent demonstration that it contributes to several aspects of adult wound repair, we examined the role of COX-2 in the fetal wound healing process. These studies demonstrate differential expression of the COX-2 enzyme in early and late gestation fetal wounds. Furthermore, PGE2, a COX-2 product shown to mediate many processes in the skin, caused a delay in healing and the production of a scar when introduced into early fetal wounds. These data further our understanding about the fundamental differences between scarless healing and normal repair, and suggest the involvement of COX-2 in the production of scar tissue (23-28).

In contrast to adult cutaneous wound repair, early gestational fetal cutaneous wounds heal by a process of regeneration, resulting in little or no scarring. Studies indicate that downregulation of HoxB13 protein, a member of the highly conserved family of Hox transcription factors, occurs during fetal scarless wound healing (29-30). No down-regulation was noted in adult wounds. When evaluating healing of adult cutaneous wounds in Hoxb13 knockout (KO) mice, tensiometry was used to measure the tensile strength of incisional wounds over a 60-day time course. Overall, Hoxb13 KO wounds are significantly stronger than wild-type (WT). Histological evaluation of incisional wounds shows that 7-day-old Hoxb13 KO wounds are significantly smaller and that 60-day-old Hoxb13 KO wounds exhibit a more normal collagen architecture compared with WT wounds. The excisional wounds close at a faster rate in Hoxb13 KO mice. Biochemical and histochemcial analyses show that Hoxb13 KO skin contains significantly elevated levels of hyaluronan. Because higher levels of hyaluronan and enhanced wound healing are characteristics of fetal skin, therefore, the conclusion is that loss of Hoxbl3 produces a more “fetal-like” state in adult skin (31).

Smad3 protein is involved in mediating intracellular signaling by members of the transforming growth factor-beta superfamily and plays a critical role in the cellular proliferation, differentiation, migration, and elaboration of matrix pivotal to cutaneous wound healing. Cross-talk between Smad3 and hormone signaling in vitro has been suggested as an important control mechanism regulating cell activities; however, its relevance in vivo is unknown. Ashcroft GS et al. reported that Smad3 plays a role in androgen-mediated inhibition of wound healing but not in the responses to estrogen modulation in vivo (30). Both wild-type and Smad3 null female mice exhibited delayed healing following ovariectomy, which could be reversed by estrogen replacement. By contrast, castration accelerated healing in wild-type male mice and was reversible by exogenous androgen treatment. Intriguingly, modulation of androgen levels resulted in no discernible perturbation in the healing response in the Smad3 null mice. Mutant monocytes could be lipopolysaccharide stimulated to produce specific pro-inflammatory agents (macrophage monocyte inhibitory factor) in a fashion similar to wild-type cells, but exhibited a muted response to androgen-mediated stimulation while maintaining a normal response to estrogen-induced macrophage inhibitory factor inhibition. These data suggest that Smad3 plays a role in mediating androgen signaling during the normal wound healing response and implicate Smad3 in the modulation of inflammatory cell activity by androgens.

Fibronectin (FN) is a multi-functional, adhesion protein and involved in multi-steps of the wound healing process. Strong evidence suggests that FN protein diversity is controlled by alternative RNA splicing; a coordinated transcription and RNA processing that is development-, age-, and tissue/cell type-regulated. Expression, regulation, and biological function of the FN gene and various spliced forms in this model are unknown. Airway and skin incisional wounds were made in fetal (gestation days 21-23), weanling (4-6 weeks) and adult (>6 months) rabbits. Expression profiles were obtained using mRNA differential display and cDNAs of interest were cloned, sequenced and validated by real-time PCR. The increased levels of both Fh1 and Sfrs3 transcripts were sustained up to 48 h in weanling airway mucosal wounds. The augmentations of the two genes in postnatal airway mucosal wounds were more prominent than that in skin wounds, indicating that the involvement of Sfrs3 and Fh1 genes in postnatal airway mucosal wounds is tissue-specific (31). Literature provides evidence that SRp20 is indeed involved in the alternative splicing of FN and that the embryonic FN variants reappear during adult wound healing. A connection between the enhanced molecular activity of Sfrs3 and the regulation of the FN gene expression through alternative splicing during the early events of postnatal airway mucosal wound repair was proposed. Dovi JV, et al. reported that accelerated wound closure in neutrophil-depleted mice was observed (32).

RNA interference (RNAi) inhibitors, the intermediate short interfering RNA oligonucleotides (siRNAs), provide a unique advantage for using combination of multiple siRNA duplexes to target multiple disease causing genes in the same treatment, since all siRNA duplexes are chemically homogenous with same source of origin and same manufacturing process (33). The inventors believe that many types of human diseases, including cancer, inflammatory conditions, autoimmune diseases and infectious diseases are able to be treated with much better clinical efficacy using such potent siRNA inhibitors with minimum toxicity and safety concerns. Based on the attractive technology of RNA interference for silencing a particular gene expression (34), siRNA therapy may represent an attractive and powerful approach in preventing scar formation in surgery or other wounds.

### SUMMARY OF THE INVENTION

This invention provides targeting polynucleotides, such as siRNA, that target inflammatory-modulatory or inflammatory-effector genes present in a cell of injured cutaneous tissue. These polynucleotides may be single-stranded linear, double-stranded linear or hairpin structures. The sequence of these targeting polynucleotides may be derived from sequences listed in tables 1-9 (see below).

This invention also provides a method of suppressing scar formation during the cutaneous wound healing process by contacting the injured tissues or cells, at a time of a surgery, wound treatment, injury recovery or skin grafting, with a composition comprising a targeting polynucleotide of the invention. In one embodiment, the composition is applied topically. In another embodiment, the composition is locally injected. The method of the invention can be effective in down-regulating or inhibiting an inflammatory-modulatory or inflammatory-effector gene during the wound healing process. The tissue contacted by the composition containing the targeting polynucleotide may be related to the cutaneous region. The tissue contacted by the composition may have been injured by burning, chemicals, laser, plastic surgery, skin grafting, surgery or physical cut. The cells contacted by the composition include, but are not limited to, epithelial cells, vascular endothelium, vascular smooth muscle cells, myocardium (heart) and passenger leukocytes resident in the cutaneous tissue at the time of wound healing. The method of the invention may be used to treat a human or a non-human mammal.

The composition used for contacting injured tissues or cells may comprise a plurality of targeting polynucleotides of the invention and the polynucleotides may target a plurality of gene sequences. The composition may further comprise a PolyTran polymer solution, a TargeTran nanoparticle solution, small molecule drugs, monoclonal antibody drugs or other immune modulators. The targeting polynucleotides found in the composition may target sequences of genes such as Cox-2, fibronectin, Hoxb13, IL-6, IL-8, Sfrs3 and TGF-β1, found in tables 1-7 (see below). The targeting polynucleotides may comprise one or more siRNA duplexes against one or more gene sequences, such as Cox-2/TGF-β1/IL-8, Cox-2/TGF-β1/IL-6, Cox-2/TGF-β2/IL-8, Cox-2/TGF-β2/IL-6, Cox-2/Hoxbl3/IL-6, Cox-2/Hoxb13/IL-8, Hoxb13/TGF-β1/Sfrs3, Cox-2/TGF-β1/fibronectin, Cox-2/TGF-β2/fibronectin, Cox-2/TGF-β1/smad3 and other combinations of three or more gene sequences. The polynucleotides of the invention may be mixed in equal or different ratios.

### BRIEF DESCRIPTION OF THE DRAWING

FIG. 1 is a schematic showing certain embodiments of the polynucleotides of the invention with targeting sequences represented by lightly shaded blocks. The length of the polynucleotides may range from 1 to 200 nucleotides. Panel A shows linear polynucleotides, and panel B shows a hairpin loop polynucleotide. Disclosed target sequences are labeled as “SEQ” while sequences complementary to these are labeled as “COMPL”. In panel A, b), the horizontal line above the polynucleotide and the darker shading surrounding the SEQ block indicate that the complete targeting sequence (TARGET) is longer than and contains the sequence represented by SEQ. In panel A, c), FRAGMENT≦15 indicates the targeting sequence ranges between 15 nucleotides and one nucleotide less than the disclosed reference sequence. In panel A, d), the darker vertical bars indicate that up to five nucleotides may differ from the disclosed reference sequence.

### DETAILED DESCRIPTION OF THE INVENTION

The present invention discloses use of one or more siRNA therapeutic agents to suppress inflammatory responses to surgical and traumatic skin wounds, thus promoting scar-free healing. The therapeutic polynucleotides are directed to one or more of the following targets: TGF-beta-1 (GenBank Accession No. CR601792), TGF-beta-2 (GenBank Accession No. Y00083), Cox-2 (GenBank Accession No. M90100), IL-6 (GenBank Accession No. M18403), IL-8 (GenBank Accession No. NM_{—}000584), Hoxb13 (GenBank Accession No. BC070233), Fibronectin (U42594), Smad3 (U68019), and Sfrs3 (GenBank Accession No. AF107405).

As used herein, “oligonucleotides” and similar terms based on this relate to short polymers composed of naturally occurring nucleotides as well as to polymers composed of synthetic or modified nucleotides, as described in the immediately preceding paragraph. Oligonucleotides may be 10 or more nucleotides in length, or 15, or 16, or 17, or 18, or 19, or 20 or more nucleotides in length, or 21, or 22, or 23, or 24 or more nucleotides in length, or 25, or 26, or 27, or 28 or 29, or 30 or more nucleotides in length, 35 or more, 40 or more, 45 or more, up to about 50, nucleotides in length. An oligonucleotide that is an siRNA may have any number of nucleotides between 15 and 30 nucleotides. In many embodiments an siRNA may have any number of nucleotides between 19 and 25 nucleotides (35).

The terms “polynucleotide” and “oligonucleotide” are used synonymously herein.

### Small Interfering RNA

According to the invention, gene expression of inflammatory-regulator or inflammatory-effector gene targets is attenuated by RNA interference. Expression products of a inflammatory-regulator or inflammatory-effector gene are targeted by specific double stranded siRNA nucleotide sequences that are complementary to at least a segment of the inflammatory-regulator or inflammatory-effector gene target sequence that contains any number of nucleotides between 15 and 30, or in many cases, contains anywhere between 21 and 25 nucleotides. The target may occur in the 5′ untranslated (UT) region, in a coding sequence, or in the 3′ UT region. See, e.g., PCT applications WO00/44895, WO99/32619, WO01/75164, WO01/92513, WO 01/29058, WO01/89304, WO02/16620, and WO02/29858, each incorporated by reference herein in their entirety.

According to the methods of the present invention, inflammatory-regulator or inflammatory-effector gene expression, and thereby scar formation due to the initial inflammatory reaction to the cutaneous tissue injury, is suppressed using siRNA. A targeting polynucleotide according to the invention includes an siRNA oligonucleotide. Such an siRNA can also be prepared by chemical synthesis of nucleotide sequences identical or similar to an intended sequence (36). Alternatively, a targeting siRNA can be obtained using a targeting polynucleotide sequence, for example, by digesting an inflammatory-regulator or inflammatory-effector ribopolynucleotide sequence in a cell-free system, such as, but not limited to, a Drosophila extract, or by transcription of recombinant double stranded cRNA.

Efficient silencing is generally observed with siRNA duplexes composed of a 16-30 nt sense strand and a 16-30 nt antisense strand of the same length. In many embodiments each strand of an siRNA paired duplex has in addition an overhang at the 3′ end that may be 1 nt, or 2 nt, or 3 nt, or 4 nt long; commonly a 3′-overhang is 2 nt long. The sequence of the 2-nt 3′ overhang makes an additional small contribution to the specificity of siRNA target recognition. In one embodiment, the nucleotides in the 3′ overhang are ribonucleotides. In an alternative embodiment, the nucleotides in the 3′ overhang are deoxyribonucleotides. Use of 3′ deoxynucleotides provides enhanced intracellular stability.

A recombinant expression vector of the invention, when introduced within a cell, is processed to provide an RNA that includes an siRNA sequence targeting an inflammatory-regulator or inflammatory-effector gene within the cell. Such a vector is a DNA molecule cloned into an expression vector comprising operatively-linked regulatory sequences flanking the inflammatory-regulator or inflammatory-effector gene targeting sequence in a manner that allows for expression. From the vector, an RNA molecule that is antisense to target RNA is transcribed by a first promoter (e.g., a promoter sequence 3′ of the cloned DNA) and an RNA molecule that is the sense strand for the RNA target is transcribed by a second promoter (e.g., a promoter sequence 5′ of the cloned DNA). The sense and antisense strands then hybridize in vivo to generate siRNA constructs targeting an inflammatory-regulator or inflammatory-effector gene sequence. Alternatively, two constructs can be utilized to create the sense and anti-sense strands of a siRNA construct. Further, cloned DNA can encode a transcript having secondary structure, wherein a single transcript has both the sense and complementary antisense sequences from the target gene or genes. In an example of this embodiment, a hairpin RNAi product is similar to all or a portion of the target gene. In another example, a hairpin RNAi product is a siRNA. The regulatory sequences flanking the inflammatory-regulator or inflammatory-effector gene sequence may be identical or may be different, such that their expression may be modulated independently, or in a temporal or spatial manner.

In certain embodiments, siRNAs are transcribed intracellularly by cloning the inflammatory-regulator or inflammatory-effector gene sequences into a vector containing, e.g., a RNA pol III transcription unit from the smaller nuclear RNA (snRNA) U6 or the human RNase P RNA H1. One example of a vector system is the GeneSuppressor™ RNA Interference kit (commercially available from Imgenex). The U6 and H1 promoters are members of the type III class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′ UU overhang in the expressed siRNA, which is similar to the 3′ overhangs of synthetic siRNAs. Any sequence less than 400 nucleotides in length can be transcribed by these promoters, therefore they are ideally suited for the expression of around 15- to 30-nucleotide siRNAs in, e.g., an approximately 50 to 100-nucleotide RNA stem loop transcript. The characteristics of RNAi and of factors affecting siRNA efficacy have been studied (37).

In a first aspect, the invention provides an isolated polynucleotide whose length can be any number of nucleotides that is 200 or fewer, and 15 or greater. The polynucleotide includes a first nucleotide sequence that targets a gene sequence present in cutaneous cells of the injured tissues and identified herein as a target. In the polynucleotide any T (thymidine) or any U (uridine) may optionally be substituted by the other. Additionally, in the polynucleotide the first nucleotide sequence consists of a) a sequence whose length is any number of nucleotides from 15 to 30, or b) a complement of a sequence given in a). Such a polynucleotide may be termed a linear polynucleotide herein. A single stranded polynucleotide frequently is one strand of a double stranded siRNA.

In a related aspect, the polynucleotide described above further includes a second nucleotide sequence separated from the first nucleotide sequence by a loop sequence, such that the second nucleotide sequence
a) has substantially the same length as the first nucleotide sequence, and
b) is substantially complementary to the first nucleotide sequence.

In this latter structure, termed a hairpin polynucleotide, the first nucleotide sequence hybridizes with the second nucleotide sequence to form a hairpin whose complementary sequences are linked by the loop sequence. A hairpin polynucleotide is digested intracellularly to form a double stranded siRNA.

In many embodiments of the linear polynucleotide and of the hairpin polynucleotide the first nucleotide sequence is either
a) a targeting sequence that targets a sequence chosen from the sequences given in Tables 1a to 9b below;
b) a targeting sequence longer than the sequence given in item a) wherein the targeting sequence targets a sequence chosen from Tables 1a-9b,
c) a fragment of a sequence given in a) or b) wherein the fragment consists of a sequence of contiguous bases at least 15 nucleotides in length and at most one base shorter than the chosen sequence,
d) a targeting sequence wherein up to 5 nucleotides differ from a sequence given in a)-c), or
e) a complement of any sequence given in a) to d).

TABLE 1
Human Cox-2 (l9mer):

| | | |
|---|---|---|
| 1. siRNA_371 | 371 | CCCUUCCUUCGAAAUGCAA |
| 2. siRNA_372 | 372 | CCUUCCUUCGAAAUGCAAU |
| 3. siRNA_468 | 468 | GCUGGGAAGCCUUCUCUAA |
| 4. siRNA_512 | 512 | CCUCCUGUGCCUGAUGAUU |
| 5. siRNA_562 | 562 | GCAGCUUCCUGAUUCAAAU |
| 6. siRNA_1102 | 1102 | GCAACACUUGAGUGGCUAU |
| 7. siRNA_1461 | 1461 | GCUUUAUGCUGAAGCCCUA |
| 8. siRNA_1602 | 1602 | CCAUCUUUGGUGAAACCAU |
| 9. siRNA_1776 | 1776 | GCUGUCCCUUUACUUCAUU |
| 10. siRNA_2853 | 2853 | CCCAAAUUAUUGGUUCCAA |

Human Cox-2 (25mer):

| | | |
|---|---|---|
| 1. stealth_394 | 394 | GAGUUAUGUGUUGACAUCCAGAUCA |
| 2. stealth_412 | 412 | CAGAUCACAUUUGAUUGACAGUCCA |
| 3. stealth_473 | 473 | GAAGCCUUCUCUAACCUCUCCUAUU |
| 4. stealth_534 | 534 | CGACUCCCUUGGGUGUCAAAGGUAA |
| 5. stealth_690 | 690 | CAGAUCAUAAGCGAGGGCCAGCUUU |
| 6. stealth_849 | 849 | CAGUCAAAGAUACUCAGGCAGAGAU |
| 7. stealth_1041 | 1041 | UCCAGACAAGCAGGCUAAUACUGAU |
| 8. stealth_1103 | 1103 | CAACACUUGAGUGGCUAUCACUUCA |
| 9. stealth 1455 | 1455 | GCAAACGCUUUAUGCUGAAGCCCUA |
| 10. stealth_1459 | 1459 | ACGCUUUAUGCUGAAGCCCUAUGAA |

TABLE 2
Human Fibronectin (19mer):

| | | |
|---|---|---|
| 1. siRNA_121 | 121 | CCUGCGAUUCACCAACAUU |
| 2. siRNA_580 | 580 | GGUCAGCAUCGUUGCUCUU |
| 3. siRNA_603 | 603 | GCAGAGAGGAAAGUCCCUU |
| 4. siRNA_719 | 719 | GCUGUCACAGUGAGAUAUU |
| 5. siRNA_801 | 801 | GCAAGUCUACAGCUACCAU |
| 6. siRNA_890 | 890 | GCAAGCAGCAAGCCAAUUU |
| 7. siRNA_894 | 894 | GCAGCAAGCCAAUUUCCAU |
| 8. siRNA_897 | 897 | GCAAGCCAAUUUCCAUUAA |
| 9. siRNA_919 | 919 | CCGAACAGAAAUUGACAAA |
| 10. siRNA_1076 | 1076 | GGUCCAGAUCAAACAGAAA |

Human Fibronectin (25mer):

| | | |
|---|---|---|
| 1. stealth_199 | 199 | CCUGGUGCGUUACUCACCUGUGAAA |
| 2. stealth_614 | 614 | AGUCCCUUAUUGAUUGGCCAACAAU |
| 3. stealth_726 | 726 | CAGUGAGAUAUUACAGGAUCACUUA |
| 4. stealth_820 | 820 | CAGCGGCCUUAAACCUGGAGUUGAU |
| 5. stealth_837 | 837 | GAGUUGAUUAUACCAUCACUGUGUA |
| 6. stealth_888 | 888 | CCGCAAGCAGCAAGCCAAUUUCCAU |
| 7. stealth_892 | 892 | AAGCAGCAAGCCAAUUUCCAUUAAU |
| 8. stealth_893 | 893 | AGCAGCAAGCCAAUUUCCAUUAAUU |
| 9. stealth_1112 | 1112 | CAGCCCACAGUGGAGUAUGUGGUUA |
| 10. stealth_1119 | 1119 | CAGUGGAGUAUGUGGUUAGUGUCUA |

TABLE 3
Human Hoxb13 (19mer):

| | | |
|---|---|---|
| 1. siRNA_99 | 99 | CCGGCAAUUAUGCCACCUU |
| 2. siRNA_126 | 126 | CCAAGGAUAUCGAAGGCUU |
| 3. siRNA_269 | 269 | CCAAAGCAAUGCCACCCAU |
| 4. siRNA_321 | 321 | CCGUGCCUUAUGGUUACUU |
| 5. siRNA_789 | 789 | GGGAGUAUGCGGCUAACAA |
| 6. siRNA_895 | 895 | GGUCAAAGAGAAGAAGGUU |
| 7. siRNA_1181 | 1181 | CCCAGUCAUAAUCAUUCAU |
| 8. siRNA_1239 | 1239 | CCAUGAUCGUUAGCCUCAU |
| 9. sIRNA_1282 | 1282 | GCACUUUAGAAACCGCUUU |
| 10. siRNA_1296 | 1296 | GCUUUCAUGAAUUGAGCUA |

Human Hoxb13 (25mer):

| | | |
|---|---|---|
| 1. stealth_332 | 332 | GGUUACUUUGGAGGCGGGUACUACU |
| 2. stealth_788 | 788 | CGGGAGUAUGCGGCUAACAAGUUCA |
| 3. stealth_791 | 791 | GAGUAUGCGGCUAACAAGUUCAUCA |
| 4. stealth_902 | 902 | GAGAAGAAGGUUCUCGCCAAGGUGA |
| 5. stealth_1167 | 1167 | CCCAAAGAACCUGGCCCAGUCAUAA |
| 6. stealth_1183 | 1183 | CAGUCAUAAUCAUUCAUCCUGACAG |
| 7. stealth_1193 | 1193 | CAUUCAUCCUGACAGUGGCAAUAAU |
| 8. stealth_1268 | 1268 | UAGAGCUCUGUAGAGCACUUUAGAA |
| 9. stealth_1280 | 1280 | GAGCACUUUAGAAACCGCUUUCAUG |
| 10. stealth_1294 | 1294 | CCGCUUUCAUGAAUUGAGCUAAUUA |

TABLE 4
Human IL-6 (19mer):

| | | |
|---|---|---|
| 1. sIRNA_250 | 250 | GCAUCUCAGCCCUGAGAAA |
| 2. siRNA_258 | 258 | GCCCUGAGAAAGGAGACAU |
| 3. sIRNA 360 | 360 | GGAUGCUUCCAAUCUGGAU |
| 4. sIRNA_364 | 364 | GCUUCCAAUCUGGAUUCAA |
| 5. siRNA_375 | 375 | GGAUUCAAUGAGGAGACUU |
| 6. siRNA_620 | 620 | GCAGGACAUGACAACUCAU |
| 7. siRNA_706 | 706 | GGCACCUCAGAUUGUUGUU |
| 8. sIRNA_768 | 768 | GCACAGAACUUAUGUUGUU |
| 9. siRNA_949 | 949 | GGAAAGUGGCUAUGCAGUU |
| 10. siRNA_950 | 950 | GAAAGUGGCUAUGCAGUUU |

Human IL-6 (25mer):

| | | |
|---|---|---|
| 1. stealth_256 | 256 | CAGCCCUGAGAAAGGAGACAUGUAA |
| 2. stealth_359 | 359 | UGGAUGCUUCCAAUCUGGAUUCAAU |
| 3. stealth_429 | 429 | GAGGUAUACCUAGAGUACCUCCAGA |
| 4. stealth_446 | 446 | CCUCCAGAACAGAUUUGAGAGUAGU |
| 5. stealth_631 | 631 | CAACUCAUCUCAUUCUGCGCAGCUU |
| 6. stealth_705 | 705 | GGGCACCUCAGAUUGUUGUUGUUAA |
| 7. stealth_762 | 762 | CACUGGGCACAGAACUUAUGUUGUU |
| 8. stealth_767 | 767 | GGCACAGAACUUAUGUUGUUCUCUA |
| 9. stealth 768 | 768 | GCACAGAACUUAUGUUGUUCUCUAU |
| 10. stealth_1002 | 1002 | UGGAAAGUGUAGGCUUACCUCAAAU |

TABLE 5
HumanIL-8 (19mer):

| | | |
|---|---|---|
| 1. siRNA_1341 | 1341 | UACUCCCAGUCUUGUCAUU |
| 2. siRNA_1342 | 1342 | ACUCCCAGUCUUGUCAUUG |
| 3. siRNA_1345 | 1345 | CCCAGUCUUGUCAUUGCCA |
| 4. siRNA_1346 | 1346 | CCAGUCUUGUCAUUGCCAG |
| 5. siRNA_1364 | 1364 | GCUGUGUUGGUAGUGCUGU |
| 6. siRNA_1371 | 1371 | UGGUAGUGCUGUGUUGAAU |
| 7. siRNA_1372 | 1372 | GGUAGUGCUGUGUUGAAUU |
| 8. siRNA_1373 | 1373 | GUAGUGCUGUGUUGAAUUA |
| 9. siRNA_1378 | 1378 | GCUGUGUUGAAUUACGGAA |
| 10. siRNA_1379 | 1379 | CUGUGUUGAAUUACGGAAU |

Human IL-8(25mer):

| | | |
|---|---|---|
| 1. stealth_1364 | 1364 | GCUGUGUUGGUAGUGCUGUGUUGAA |
| 2. stealth_1366 | 1366 | UGUGUUGGUAGUGCUGUGUUGAAUU |
| 3. stealth_1372 | 1372 | GGUAGUGCUGUGUUGAAUUACGGAA |
| 4. stealth_1374 | 1374 | UAGUGCUGUGUUGAAUUACGGAAUA |
| 5. stealth_1375 | 1375 | AGUGCUGUGUUGAAUUACGGAAUAA |
| 6. stealth_1377 | 1377 | UGCUGUGUUGAAUUACGGAAUAAUG |
| 7. stealth_1378 | 1378 | GCUGUGUUGAAUUACGGAAUAAUGA |

TABLE 6
Human Sfrs3 (19mer):

| | | |
|---|---|---|
| 1. siRNA_28 | 28 | GGAAAGCGGGAAGACUCAU |
| 2. sIRNA_109 | 109 | CCUGUCCAUUGGACUGUAA |
| 3. siRNA_114 | 114 | CCAUUGGACUGUAAGGUUU |
| 4. siRNA_509 | 509 | GCUGUCUCGGGAGAGAAAU |
| 5. siRNA_612 | 612 | GGUGUACAGGAAAUUACUU |
| 6. siRNA_749 | 749 | GGUGUAAUUCUCUAUGGUU |
| 7. siRNA_785 | 785 | GGCAUGUAAUACCAAGAAU |
| 8. siRNA_1452 | 1452 | CCUAUUGGAAGCCAUACUU |
| 9. siRNA 1612 | 1612 | GGCACUAUGGAUUAGUCUU |
| 10. siRNA_1976 | 1976 | GCAGGUGUUGUAAUUUCAA |

Human Sfrs3 (25mer):

| | | |
|---|---|---|
| 1. stealth_82 | 82 | CCCUAGAUCUCGAAAUGCAUCGUGA |
| 2. stealth_108 | 108 | UCCUGUCCAUUGGACUGUAAGGUUU |
| 3. stealth_109 | 109 | CCUGUCCAUUGGACUGUAAGGUUUA |
| 4. stealth_558 | 558 | CGUAGUCGAUCUAGGUCAAAUGAAA |
| 5. stealth_601 | 601 | GCAAGAGAAGUGGUGUACAGGAAAU |
| 6. stealth_743 | 743 | CACAAAGGUGUAAUUCUCUAUGGUU |
| 7. stealth_1422 | 1422 | GAGCUUGGUACCAAGUCCAGGUAUA |
| 8. stealth_1448 | 1448 | CAUUCCUAUUGGAAGCCAUACUUAU |
| 9. stealth_1567 | 1567 | AAGCAGUUGGUUACACGAUUCUUAU |
| 10. stealth_1611 | 1611 | AGGCACUAUGGAUUAGUCUUCUGAA |

TABLE 7
Human TGF-b1 (19mer):

| | | |
|---|---|---|
| 1. siRNA_1380 | 1380 | CCAGAAAUACAGCAACAAU |
| 2. siRNA_1391 | 1391 | GCAACAAUUCCUGGCGAUA |
| 3. siRNA 1538 | 1538 | CCUGUGACAGCAGGGAUAA |
| 4. siRNA_1569 | 1569 | GGACAUCAACGGGUUCACU |
| 5. siRNA_1610 | 1610 | CCACCAUUCAUGGCAUGAA |
| 6. siRNA_1631 | 1631 | GGCCUUUCCUGCUUCUCAU |
| 7. siRNA_1702 | 1702 | GCCCUGGACACCAACUAUU |
| 8. siRNA 1754 | 1754 | GGCAGCUGUACAUUGACUU |
| 9. siRNA-1888 | 1888 | GCCCUGUACAACCAGCAUA |
| 10. siRNA_1889 | 1889 | CCCUGUACAACCAGCAUAA |

Human TGF-b1 (25mer):

| | | |
|---|---|---|
| 1. stealth_1363 | 1363 | CAGCACGUGGAGCUGUACCAGAAAU |
| 2. stealth_1366 | 1366 | CACGUGGAGCUGUACCAGAAAUACA |
| 3. stealth_1372 | 1372 | GAGCUGUACCAGAAAUACAGCAACA |
| 4. stealth_1435 | 1435 | AGCGACUCGCCAGAGUGGUUAUCUU |
| 5. stealth_1436 | 1436 | GCGACUCGCCAGAGUGGUUAUCUUU |
| 6. stealth_1547 | 1547 | GCAGGGAUAACACACUGCAAGUGGA |
| 7. stealth_1558 | 1558 | ACACUGCAAGUGGACAUCAACGGGU |
| 8. stealth_1564 | 1564 | CAAGUGGACAUCAACGGGUUCACUA |
| 9. stealth 1625 | 1625 | UGAACCGGCCUUUCCUGCUUCUCAU |
| 10. stealth_1708 | 1708 | GACACCAACUAUUGCUUCAGCUCCA |

TABLE 8
Human TGF-b2 (19mer):

| | | |
|---|---|---|
| 1. siRNA_249 | 249 | CCUGCAGCACACUCGAUAU |
| 2. siRNA_727 | 727 | GCGCUACAUCGACAGCAAA |
| 3. siRNA_1088 | 1088 | GCUUUGGAUGCGGCCUAUU |
| 4. siRNA_1093 | 1093 | GGAUGCGGCCUAUUGCUUU |
| 5. siRNA_1131 | 1131 | GCUGCCUACGUCCACUUUA |
| 6. siRNA_1134 | 1134 | GCCUACGUCCACUUUACAU |
| 7. siRNA_1135 | 1135 | CCUACGUCCACUUUACAUU |
| 8. siRNA_1194 | 1194 | CCAAAGGGUACAAUGCCAA |
| 9. siRNA_1267 | 1267 | GGUCCUGAGCUUAUAUAAU |
| 10. siRNA_1317 | 1317 | GCUGCGUGUCCCAAGAUUU |

Human TGF-b2 (25mer):

| | | |
|---|---|---|
| 1. stealth_697 | 697 | CAAGUCCAAAGAUUUAACAUCUCCA |
| 2. stealth_784 | 784 | CGAUGUAACUGAUGCUGUUCAUGAA |
| 3. stealth_916 | 916 | ACUAGAAGCAAGAUUUGCAGGUAUU |
| 4. stealth_1162 | 1162 | GAGGGAUCUAGGGUGGAAAUGGAUA |
| 5. stealth_1193 | 1193 | CCCAAAGGGUACAAUGCCAACUUCU |
| 6. stealth_1204 | 1204 | CAAUGCCAACUUCUGUGCUGGAGCA |
| 7. stealth_1267 | 1267 | GGUCCUGAGCUUAUAUAAUACCAUA |
| 8. stealth_1321 | 1321 | CGUGUCCCAAGAUUUAGAACCUCUA |
| 9. stealth_1327 | 1327 | CCAAGAUUUAGAACCUCUAACCAUU |
| 10. stealth_1371 | 1371 | CACCCAAGAUUGAACAGCUUUCUAA |

TABLE 9
Targeting Smad3 siRNA (19mer):

| | | |
|---|---|---|
| 1. siRNA_176 | 176 | GCCUGGUCAAGAAACUCAA |
| 2. siRNA_428 | 428 | GCGUGAAUCCCUACCACUA |
| 3. siRNA_822 | 822 | GCCAUCCAUGACUGUGGAU |
| 4. siRNA_827 | 827 | CCAUGACUGUGGAUGGCUU |
| 5. sIRNA_1079 | 1079 | GCAACCUGAAGAUCUUCAA |
| 6. siRNA_1182 | 1182 | CCGCAUGAGCUUCGUCAAA |
| 7. sIRNA_1250 | 1250 | GGAUUGAGCUGCACCUGAA |
| 8. sIRNA 1325 | 1325 | GCUGUUCCAGUGUGUCUUA |
| 9. siRNA:1411 | 1411 | GGAACUCUACUCAACCCAU |
| 10. siRNA1540 | 1540 | CCAAACACAUUUACCCUUU |

Targeting Smad3 siRNA (25mer):

| | | |
|---|---|---|
| 1. stealth_447 | 447 | CCAGAGAGUAGAGACACCAGUUCUA |
| 2. stealth_632 | 632 | GAGAAACCAGUGACCACCAGAUGAA |
| 3. stealth_707 | 707 | CAGCACAUAAUAACUUGGACCUGCA |
| 4. stealth_1070 | 1070 | CACCAGGAUGCAACCUGAAGAUCUU |
| 5. stealth_1331 | 1331 | CCAGUGUGUCUUAGAGACAUCAAGU |
| 6. stealth_1332 | 1332 | CAGUGUGUCUUAGAGACAUCAAGUA |
| 7. stealth_1444 | 1444 | AAGAAAUCUUUCUCCCUCAACUGAA |
| 8. stealth_1499 | 1499 | CGAGCAAACCCAGAGGUGGAUGUUA |
| 9. stealth_1500 | 1500 | GAGCAAACCCAGAGGUGGAUGUUAU |
| 10. stealth_1511 | 1511 | GAGGUGGAUGUUAUGAACAGCUGUG |

In various embodiments of a linear polynucleotide or a hairpin polynucleotide the length of the first nucleotide sequence is any number of nucleotides from 21 to 25. In many embodiments a linear polynucleotide or a hairpin polynucleotide consists of a targeting sequence that targets a sequence chosen from Tables 1a-9b, and optionally includes a dinucleotide overhang bound to the 3′ of the chosen sequence. In yet additional embodiments of a linear polynucleotide or a hairpin polynucleotide the dinucleotide sequence at the 3′ end of the first nucleotide sequence is TT, TU, UT, or UU and includes either ribonucleotides or deoxyribonucleotides or both. In various further embodiments a linear or hairpin polynucleotide may be a DNA, or it may be an RNA, or it may be composed of both deoxyribonucleotides and ribonucleotides.

In an additional aspect the invention provides a double stranded polynucleotide that includes a first linear polynucleotide strand described above and a second polynucleotide strand that is complementary to at least the first nucleotide sequence of the first strand and is hybridized thereto to form a double stranded siRNA composition.

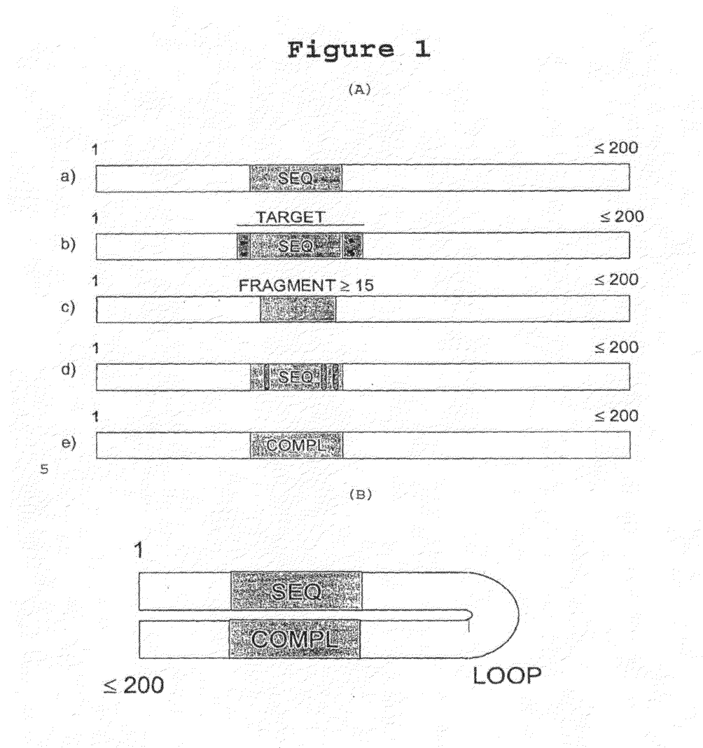

FIG. 1 provides schematic representations of certain embodiments of the polynucleotides of the invention. The invention discloses target sequences, or in certain cases siRNA sequences that are slightly mismatched from a target sequence, all of which are provided in Tables 1a-9b. The sequences disclosed therein range in length from 19 nucleotides to 25 nucleotides. The targeting sequences are represented by the lightly shaded blocks in FIG. 1. FIG. 1, Panel A, a) illustrates an embodiment in which the disclosed sequence shown as “SEQ” may optionally be included in a larger polynucleotide whose overall length may range up to 200 nucleotides.

The invention additionally provides that, in the targeting polynucleotide, a targeting sequence directed to a target sequence chosen from Tables 1a-9b may be part of a longer targeting sequence such that the targeting polynucleotide targets a sequence that is longer than the first nucleotide sequence represented by SEQ. This is illustrated in FIG. 1, Panel A, b), in which the complete targeting sequence is shown by the horizontal line above the polynucleotide, and by the darker shading surrounding the SEQ block. As in all embodiments of the polynucleotides, this longer sequence may optionally be included in a still larger polynucleotide of length 200 or fewer bases (FIG. 1, Panel A, b)).

The invention further provides a targeting sequence that is a fragment of any of the above targeting sequences such that the fragment targets a sequence given in Tables 1a-9b that is at least 15 nucleotides in length (and at most 1 base shorter than the reference sequence; illustrated in FIG. 1, Panel A, c)), as well as a targeting sequence wherein up to 5 nucleotides may differ from being complementary to the target sequence given in Tables 1a-9b (illustrated in FIG. 1, Panel A, d), showing, in this example, three variant bases represented by the three darker vertical bars).

Still further the invention provides a sequence that is a complement to any of the above-described sequences (shown in FIG. 1, Panel A, e), and designated as “COMPL”). Any of these sequences are included in the oligonucleotides or polynucleotides of the invention. Any linear polynucleotide of the invention may be constituted of only the sequences described in a)-e) above, or optionally may include additional bases up to the limit of 200 nucleotides. Since RNA interference requires double stranded RNAs, the targeting polynucleotide itself may be double stranded, including a second strand complementary to at least the sequence given by Tables 1a-9b and hybridized thereto, or intracellular processes may be relied upon to generate a complementary strand.

Thus the polynucleotide may be single stranded, or it may be double stranded. In still further embodiments, the polynucleotide contains only deoxyribonucleotides, or it contains only ribonucleotides, or it contains both deoxyribonucleotides and ribonucleotides. In important embodiments of the polynucleotides described herein the target sequence consists of a sequence that may be either 15 nucleotides (nt), or 16 nt, or 17 nt, or 18 nt, or 19 nt, or 20 nt, or 21 nt, or 22 nt, or 23 nt, or 24 nt, or 25 nt, or 26 nt, or 27 nt, or 28 nt, or 29, or 30 nt in length. In still additional advantageous embodiments the targeting sequence may differ by up to 5 bases from complementarity to a target sequence.

In several embodiments of the invention, the polynucleotide is an siRNA consisting of the targeting sequence with optional inclusion of a 3′ dinucleotide overhang described herein.

Alternatively, in recognition of the need for a double stranded RNA in RNA interference, the oligonucleotide or polynucleotide may be prepared to form an intramolecular hairpin looped double stranded molecule. Such a molecule is formed of a first sequence described in any of the embodiments of the preceding paragraphs followed by a short loop sequence, which is then followed in turn by a second sequence that is complementary to the first sequence. Such a structure forms the desired intramolecular hairpin. Furthermore, this polynucleotide is disclosed as also having a maximum length of 200 nucleotides, such that the three required structures enumerated may be constituted in any oligonucleotide or polynucleotide having any overall length of up to 200 nucleotides. A hairpin loop polynucleotide is illustrated in FIG. 1, Panel B.

Improving Local siRNA Delivery with Various Formulations

The present invention provides methods for prevention of scar formation during the wound healing Process due to the inflammatory reaction to the cutaneous tissue injury, by silencing or down-regulation of a target gene expression by introducing RNA interference (siRNA). In a method of the present invention, siRNA or siRNA cocktail is applied or administrated to an area of cutaneous wound during the healing process. This treatment may be provided for a human, or a non-human mammal. Because of the recent advances in cellular and molecular biology, we have greatly expanded our understanding of the biologic processes involved in wound repair and tissue regeneration and have led to improvements in wound care. Wound healing is a dynamic, interactive proces involving soluble mediators, blood cells, extracellular matrix, and parenchymal cells, and many gene functions in those cells. We believe that using siRNA inhibitors to regulate the expression of certain inflammatory activators, such as, TGF-β1,2, Cox-2, IL-6, IL-8, Hoxb13, Fibronectin, Smad3 and Sfrs3, etc, either used individually or in combination will lead to an ideal wound healing process with less scar formation. This siRNA-mediated treatment with optimum regimen will minimize the inflammation, enhance skin tissue formation and tissue remodeling, which are three major steps of the wound healing process.

### Pharmaceutical Compositions

As used herein, “pharmaceutically acceptable carrier”, referring to a pharmaceutical composition, is intended to include any and all solvents, dispersion media, coatings, antibacterial and antifungal agents, isotonic and absorption delaying agents, and the like, compatible with pharmaceutical administration. Suitable carriers are described in textbooks such as Remington's Pharmaceutical Sciences, Gennaro A R (Ed.) 20^{th} edition (2000) Williams & Wilkins Pa., USA, and Wilson and Gisvold's Textbook of Organic Medicinal and Pharmaceutical Chemistry, by Delgado and Remers, Lippincott-Raven., which are incorporated herein by reference. Preferred examples of components that may be used in such carriers or diluents include, but are not limited to, water, saline, phosphate salts, carboxylate salts, amino acid solutions, Ringer's solutions, dextrose (a synonym for glucose) solution, and 5% human serum albumin. By way of nonlimiting example, dextrose may used as 5% or 10% aqueous solutions. Liposomes and non-aqueous vehicles such as fixed oils may also be used. The use of such media and agents for pharmaceutically active substances is well known in the art. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral, nasal, inhalation, transdermal (topical), transmucosal, and rectal administration. Solutions or suspensions used for parenteral, intravenous, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose.

For administration by inhalation, the compounds are delivered in the form of an aerosol spray from pressured container or dispenser which contains a suitable propellant, e.g., a gas such as carbon dioxide, or a nebulizer.

In one embodiment, the active compounds are prepared with carriers that will protect the compound against rapid elimination from the body, such as a controlled release formulation, including implants and microencapsulated delivery systems. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release pharmaceutical active agents over shorter time periods. Advantageous polymers are biodegradable, or biocompatible. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811. Sustained-release preparations having advantageous forms, such as microspheres, can be prepared from materials such as those described above.

The siRNA polynucleotides of the invention can be inserted into vectors and used as gene therapy vectors. Gene therapy vectors can be delivered to a subject by any of a number of routes, e.g., as described in U.S. Pat. Nos. 5,703,055. Delivery can thus also include, e.g., intravenous injection, local administration (see U.S. Pat. No. 5,328,470) or stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a kit, e.g., in a container, pack, or dispenser together with instructions for administration.

A variety of carriers served to prepare formulations or pharmaceutical compositions containing siRNA inhibitors can be used to improve the local delivery of the siRNA therapeutic, through topical application, local injection or transdermal administration. In several embodiments the siRNA polynucleotides of the invention are delivered into cells in culture or into cells of interest by liposome-mediated transfection, for example by using commercially available reagents or techniques, e.g., Oligofectamine™, LipofectArnine™ reagent, LipofectAmine 2000™ (Invitrogen), as well as by electroporation, and similar techniques. The pharmaceutical compositions containing the siRNAs include additional components that protect the stability of siRNA, prolong siRNA lifetime, potentiate siRNA function, or target siRNA to specific tissues/cells. These include a variety of biodegradable polymers, cationic polymers (such as polyethyleneimine), cationic copolypeptides such as histidine-lysine (HK) polypeptides see, for example, PCT publications WO 01/47496 to Mixson et al., WO 02/096941 to Biomerieux, and WO 99/42091 to Massachusetts Institute of Technology), PEGylated cationic polypeptides, and ligand-incorporated polymers, etc. positively charged polypeptides, PolyTran solutions (saline or aqueous solution of HK polymers and polysaccharides such as natural polysaccharides, also known as scleroglucan), TargeTran (a saline or aqueous suspension of nano-particle composed of conjugated RGD-PEG-PEI polymers including a targeting ligand), surfactants (Infasurf; Forest Laboratories, Inc.; ONY Inc.), and cationic polymers (such as polyethyleneimine) (37-39). Infasurf® (calfactant) is a natural lung surfactant isolated from calf lung for use in intratracheal instillation; it contains phospholipids, neutral lipids, and hydrophobic surfactant-associated proteins B and C. The polymers can either be uni-dimensional or multi-dimensional, and also could be microparticles or nanoparticles with diameters less than 20 microns, between 20 and 100 microns, or above 100 micron (40-42). The said polymers could carry ligand molecules specific for receptors or molecules of special tissues or cells, thus be used for targeted delivery of siRNAs. The siRNA polynucleotides are also delivered by cationic liposome based carriers, such as DOTAP, DOTAP/Cholesterol (Qbiogene, Inc.) and other types of lipid aqueous solutions. Natural cream containing the siRNA inhibitors is able to topically applied to the wound tissue surface to enhance scarless wound healing.

RT-PCR to Evaluate siRNA-Mediated Gene Knockdown

To evaluate the gene knockdown efficiency of the siRNA inhibitors in vitro and in vivo, a well established the method is using Quantitative Reverse transcription PCR (QRT-PCR) to measure the mRNA levels before and after siRNA treatments. In various embodiments, the primers useful for QRT-PCR were designed specifically for measurement of mRNA levels of TGF-β1,2, Cox-2, IL-6, IL-8, Hoxb13, Fibronectin, Smad3 and Sfrs3, etc., from total RNA samples collected from cell culture experiments and skin tissues samples of mouse, rabbit and swine models.

Primers for QRT-PCR measurement of TGF-β1 mRNA:

Reverse transcription primer (1289-1268):
5′-CGGAGCTCTGATGTGTTGAAGA-3′
Upstream primer (881-902):
5′-GGCTGCGGCTGCTGCCGCTGCT-3′
Downstream primer (1181-1160):
5′-GCGTAGTAGTCGGCCTCAGGCT-3′

Primers for QRT-PCR measurement of TGF-β2 mRNA:

Reverse transcription primer (868-846):
5′-GCAGCAGGGACAGTGTAAGCTT-3′
Upstream primer (440-461):
5′-GCCGCCTGCGAGCGCGAGAGGA -3′
Downstream primer (742-721):
5′-GCTGTCGATGTAGCGCTGGGTT-3′

Primers for QRT-PCR measurement of Cox-2 mRNA:

Reverse transcription primer (1012-991):
5′-CTCCTGTTTAAGCACATCGCAT-3′
Upstream primer (564-585):
5′-GCTTCCTGATTCAAATGAGATT -3′
Downstream primer (835-814):
5′-CTCTCCATCAATTATCTGATAT-3′.

Primers for QRT-PCR measurement of Hoxb13 mRNA:

Reverse transcription primer (1150-1138):
5′-GCTGTCACATGGGGTTCCGTCT-3′
Upstream primer (701-722):
5′-GGGCAGCACCCTCCTGACGCCT-3′
Downstream primer (1025-1004):
5′CCCAGCCTGGGCTTGGCAGGTT -3′.

Primers for QRT-PCR measurement of Fibronectin mRNA:

Reverse transcription primer (1032-1011):
5′-GTGGTTACTCTGTAACCAGTAA-3′
Upstream primer (561-582):
5′- CTCCAGGCACAGAGTATGTGGT-3′
Downstream primer (872-860):
5′-CAGTGACAGCATACACAGTGAT-3′.

Primers for QRT-PCR measurement of Sfrs3 mRNA:

Reverse transcription primer (1060-1039):
5′-GCAGCATTTCGTTTTCCCTGAT -3′
Upstream primer (571-592):
5′-GGTCAAATGAAAGGAAATAGAA-3′
Downstream primer (880-859):
5′-GGTTTATTATCAGTCTGTGCAT-3′.

Primers for QRT-PCR measurement of IL-6 mRNA:

Reverse transcription primer (965-986):
5′-CTGCATAGCCACTTTCCATTAT-3′
Upstream primer (301-322):
5′-GCAGCAAAGAGGCACTGGCAGA-3′
Downstream primer (599-621):
5′-CAGCTTCGTCAGCAGGCTGGCA-3′.

Primers for QRT-PCR measurement of IL-8 mRNA:

Reverse transcription primer (870-848):
5′-GGGTTGCCAGATTTAACAGAAA-3′
Upstream primer (428-449):
5′-GAATCAGTGAAGATGCCAGTGA-3′
Downstream primer (744-723):
5′-CCTGAAATTAAAGTTCGGAT -3′.

Primers for QRT-PCR measurement of Smad3 mRNA:

Reverse transcription primer (910-888):
5′-CTGCATTCCTGTTGACATTGGA -3′
Upstream primer (310-332):
5′-GGGCTCCCTCATGTCATCTACT-3′
Downstream primer (781-759):
5′-CGTAGTAGGAGATGGAGCACCA-3′.

siRNA Cocktail Composition

In addition to using the siRNA duplex to target each of the particular gene target, such as TGF-β1,2, Cox-2, IL-6, IL-B, Hoxb13, Fibronectin, Smad3 and Sfrs3, etc., with local application either topically or subcutaneously using injectable solutions or creams, for improvement of cutaneous wound healing without scar formation, the present invention provides concepts, methods and compositions for using siRNA oligo cocktail (siRNA-OC), selectively targeting three or more those genes, as therapeutic agent for better clinical outcome of scarless wound healing. This siRNA oligo cocktail contains at least three duplexes targeting at least three mRNA targets. The present invention is based on two important aspects: first, the siRNA duplex is a very potent gene expression inhibitor, and each siRNA molecule is made of a short double-stranded RNA oligo (21-23 nt, or 24-25 nt, or 26-29 nt) with the same chemical property; Second, the cutaneous wound healing process involves soluble mediators, blood cells, extracellular matrix, and parenchymal cells, with multiple factors functioning in the inflammation, tissue formation, and tissue remodeling. Therefore, using siRNA-OC targeting multiple disease causing genes represents an advantageous therapeutic approach, due to the chemical uniformity of siRNA duplexes and synergistic effect from down regulation of multiple disease causing genes. The invention defines that siRNA-OC is a combination of siRNA duplexes targeting at least three genes, at various proportions, in various physical forms (solution or powder), and being applied through the same route at the same time, or different routes and times (such as during injury recovery) into diseased tissues.

The wound healing process can be characterized as three phases—inflammation, tissue formation, and tissue remodeling.

Tissue injury causes the disruption of blood vessels and extravasation of blood constituents. Numerous vasoactive mediators and chemotactic factors are generated by the coagulation and activated-complement pathways and by injured or activated parenchymal cells. These substances recruit inflammatory leukocytes to the site of injury. Infiltrating neutrophils cleanse the wounded area of foreign particles and bacteria and are then extruded with the eschar or phagocytosed by macrophages. In response to specific chemoattractants, such as fragments of extracellular-matrix protein, transforming growth factor b, and monocyte chemoattractant protein 1, monocytes also infiltrate the wound site and become activated macrophages that release growth factors such as platelet-derived growth factor and vascular endothelial growth factor, which initiate the formation of granulation tissue. Macrophages bind to specific proteins of the extracellular matrix by their integrin receptors, an action that stimulates phagocytosis of microorganisms and fragments of extracellular matrix by the macrophages. Adherence to the extracellular matrix also stimulates monocytes to undergo metamorphosis into inflammatory or reparative macrophages. Adherence induces monocytes and macrophages to express colony-stimulating factor 1, a cytokine necessary for the survival of monocytes and macrophages; tumor necrosis factor a, a potent inflammatory cytokine; and platelet-derived growth factor, a potent chemoattractant and mitogen for fibroblasts. Other important cytokines expressed by monocytes and macrophages are transforming growth factor a, interleukin-1, transforming growth factor b, and insulinlike growth factor I. The monocyte- and macrophage-derived growth factors are almost certainly necessary for the initiation and propagation of new tissue formation in wounds, because macrophage depleted animals have defective wound repair. Thus, macrophages appear to have a pivotal role in the transition between inflammation and repair. Clearly, the initial inflammatory phase involves multiple factors, especially those pro-inflammatory cytokines and growth factors. Therefore, down regulating those pro-inflammatory cytokines and growth factors responsible for scar formation, such as TGF-β1, 2, IL-6, IL-8 and Cox-2, using siRNA oligo cocktail (in combination) will be very beneficial. The combinations include, but are not limited to, TGF-β1/Hoxb13/IL-8, TGF-32/Hoxb13/IL-8, TGF-β1/Sfrs3/IL-8, TGF-β1/Cox-2/1L-8, TGF-β2/Hoxb13/IL-8, and TGF-β1/Smad3/IL-6.

The mixture (cocktail) can be made from all 19 mer or all 25 mer or either 19 or 25 mer oligos.

The mixture (cocktail) can be made from oligos targeting any 2 genes, or any 3 genes, or any 4 genes, or any 5 genes, or more, chosen from among the gene targets identified herein.

The siRNA cocktail can be applied with other medications, such as antibiotics, antibodies, small molecule inhibitors, cortisones, natural creams, herbal creams and other anti-inflammatory agents.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention. All publications and other references mentioned herein are incorporated by reference in their entirety. In case of conflict, the present specification, including definitions, will control. Although a number of documents are cited herein, this citation does not constitute an admission that any of these documents forms part of the common general knowledge in the art. Throughout this specification and claims, the word “comprise,” or variations such as “comprises” or “comprising” will be understood to imply the inclusion of a stated integer or group of integers but not the exclusion of any other integer or group of integers. The materials, methods, and examples are illustrative only and not intended to be limiting.

### Example 1

### Combination TGF-β1/TGF-β2/Cox-2

The following three oligonucleotides targeting the indicated sequences are combined in equal amounts (w/w/w):

siRNA sequence (T1-19-1): CCCUUCCUUCGAAAUGCAA,
targeting Cox-2,
siRNA sequence (T7-19-1): CCAGAAAUACAGCAACAAU,
targeting TGF-β1,
siRNA sequence (T8-19-1): CCUGCAGCACACUCGAUAU,
targeting TGF-β2.

They are prepared in an aqueous solution or formulated in an appropriate carrier for topical application or subcutaneous injection. Alternatively, the three targeting oligonucleotides identified above are combined in various different ratios in solution or formulated in an appropriate carrier for topical application or subcutaneous injection to promote beneficial minimization of scar formation. More generally, other siRNA oligonucleotides, targeting other sequences of the same three genes, are mixed as the cocktail for therapeutic application. Such targeting oligonucleotides include siRNAs targeting Cox2 sequences from Table 1.1 and Table 1.2; targeting TGF-β1 sequences from Table 7.1. and Table 7.2., and targeting TGF-β2 sequences from Table 8.1. and Table 8.2.

### Example 2

### Combination TGF-β1/Hoxb13/Cox-2

The following three oligonucleotides targeting the indicated sequences are combined in equal amounts (w/w/w):

siRNA sequence (T1-19-1): CCCUUCCUUCGAAAUGCAA,
targeting Cox-2,
siRNA sequence (T7-19-1): CCAGAAAUACAGCAACAAU,
targeting TGF-β1,
siRNA sequence (T3-19-1): CCGGCAAUUAUGCCACCUU,
targeting Hoxb13.

They are prepared in an aqueous solution or formulated in an appropriate carrier for topical application or subcutaneous injection. Alternatively, the three targeting oligonucleotides identified above are combined in various different ratios in solution or formulated in an appropriate carrier for topical application or subcutaneous injection to promote beneficial minimization of scar formation. More generally, other siRNA oligonucleotides, targeting other sequences of the same three genes, are mixed as the cocktail for therapeutic application. Such targeting oligonucleotides include siRNAs targeting Cox2 sequences from Table 1.1 and Table 1.2; targeting TGF-β1 sequences from Table 7.1. and Table 7.2., and targeting Hoxb13 sequences from Table 3.1. and Table 3.2.

### Example 3

### Combination TGF-□1/Hoxb13/Sfrs3

The following three oligonucleotides targeting the indicated sequences are combined in equal amounts (w/w/w):

siRNA sequence (T6-19-1): GGAAAGCGGGAAGACUCAU,
targeting Sfrs3,
siRNA sequence (T7-19-1): CCAGAAAUACAGCAACAAU,
targeting TGF-β1,
siRNA sequence (T3-19-1): CCGGCAAUUAUGCCACCUU,
targeting Hoxb13.

They are prepared in an aqueous solution or formulated in an appropriate carrier for topical application or subcutaneous injection. Alternatively, the three targeting oligonucleotides identified above are combined in various different ratios in solution or formulated in an appropriate carrier for topical application or subcutaneous injection to promote beneficial minimization of scar formation. More generally, other siRNA oligonucleotides, targeting other sequences of the same three genes, are mixed as the cocktail for therapeutic application. Such targeting oligonucleotides include siRNAs targeting Sfrs3 sequences from Table 6.1 and Table 6.2; targeting TGF-β1 sequences from Table 7.1. and Table 7.2., and targeting Hoxb13 sequences from Table 3.1. and Table 3.2.

### Example 4

### Combination TGF-β1/Hoxb13/IL-6

The following three oligonucleotides targeting the indicated sequences are combined in equal amounts (w/w/w):

siRNA sequence (T4-19-1): GCAUCUCAGCCCUGAGAAA,
targeting IL-6,
siRNA sequence (T7-19-1): CCAGAAAUACAGCAACAAU,
targeting TGF-β1,
siRNA sequence (T3-19-1): CCGGCAAWAUGCCACCUU,
targeting Hoxb13,

They are prepared in an aqueous solution or formulated in an appropriate carrier for topical application or subcutaneous injection. Alternatively, the three targeting oligonucleotides identified above are combined in various different ratios in solution or formulated in an appropriate carrier for topical application or subcutaneous injection to promote beneficial minimization of scar formation. More generally, other siRNA oligonucleotides, targeting other sequences of the same three genes, are mixed as the cocktail for therapeutic application. Such targeting oligonucleotides include siRNAs targeting IL-6 sequences from Table 4.1 and Table 4.2; targeting TGF-β1 sequences from Table 7.1. and Table 7.2., and targeting Hoxb13 sequences from Table 3.1. and Table 3.2.

### LITERATURE

1. Singer A J, Clark R A: Cutaneous wound healing. N Engl J Med 1999, 341:738-746.
2. Martin P: Wound healing: aiming for perfect skin regeneration. Science 1997, 276:75-81.
3. Rowlatt U: Intrauterine wound healing in a 20 week human fetus. Virchows Arch A Pathol Anat Histol 1979, 381:353-361.
4. Lin R Y, et al. Exogenous transforming growth factor-beta amplifies its own expression and induces scar formation in a model of human fetal skin repair. Ann Surg 1995, 222:146-154.
5. Cowin A J, et al. Expression of TGF-beta and its receptors in murine fetal and adult dermal wounds. Eur J Dermatol 2001, 11:424-431.
6. Krummel T M, et al. Transforming growth factor beta (TGF-beta) induces fibrosis in a fetal wound model. J Pediatr Surg 1988, 23:647-652.
7. Lanning D A, et al. TGF-betal alters the healing of cutaneous fetal excisional wounds. J Pediatr Surg 1999, 34:695-700.
8. Soo C, et al. Ontogenetic transition in fetal wound transforming growth factor-beta regulation correlates with collagen organization. Am J Pathol 2003, 163:2459-2476.
9. Sullivan K M, et al. A model of starless human fetal wound repair is deficient in transforming growth factor beta. J Pediatr Surg 1995, 30:198-202202-193.
10. Stelnicki E J, et al. A new in vivo model for the study of fetal wound healing. Ann Plast Surg 1997, 39:374-380.
11. Whitby D J and Ferguson M W: Immunohistochemical localization of growth factors in fetal wound healing. Dev Biol 1991, 147:207-215.
12. Roberts A B, et al. Transforming growth factor type beta: rapid induction of fibrosis and angiogenesis in vivo and stimulation of collagen formation in vitro. Proc Natl Acad Sci USA 1986, 83:4167-4171.
13. Shah M, et al. Neutralising antibody to TGF-beta 1, 2 reduces cutaneous scarring in adult rodents. J Cell Sci 1994, 107:1137-1157.
14. Shah M, et al. Control of scarring in adult wounds by neutralising antibody to transforming growth factor beta. Lancet 1992, 339:213-214.
15. Choi B M, et al. Control of scarring in adult wounds using antisense transforming growth factor-beta 1 oligodeoxynucleotides. Immunol Cell Biol 1996, 74:144-150.
16. Haynes J H, et al. Platelet-derived growth factor induces fetal wound fibrosis. J Pediatr Surg 1994, 29:1405-1408.
17. Liechty K W, et al. Diminished interleukin 6 (IL-6) production during scarless human fetal wound repair. Cytokine 2000, 12:671-676.
18. Liechty K W, et al. Diminished interleukin-8 (IL-8) production in the fetal wound healing response. J Surg Res 1998, 77:80-84.
19. Wu K K: Cyclooxygenase 2 induction: molecular mechanism and pathophysiologic roles. J Lab Clin Med 1996, 128:242-245.
20. Wilgus T A, et al. Topical application of a selective cyclooxygenase inhibitor suppresses UVB mediated cutaneous inflammation. Prostaglandins Other Lipid Mediat 2000, 62:367-384.
21. Sun W H, et al. Cyclooxygenase-2 inhibitors suppress epithelial cell kinetics and delay gastric wound healing in rats. J Gastroenterol Hepatol 2000, 15:752-761.
22. Guo J S, et al. Antiangiogenic effect of a highly selective cyclooxygenase-2 inhibitor on gastric ulcer healing in rats. Toxicol Appl Pharmacol 2002, 183:41-45.
23. Simon A M, et al. Cyclooxygenase 2 function is essential for bone fracture healing. J Bone Miner Res 2002, 17:963-976.
24. Kampfer H, et al. Cyclooxygenase-1-coupled prostaglandin biosynthesis constitutes an essential prerequisite for skin repair. J Invest Dermatol 2003, 120:880-890.
25. Blomme E A, et al. Selective cyclooxygenase-2 inhibition does not affect the healing of cutaneous full-thickness incisional wounds in SKH-1 mice. Br J Dermatol 2003, 148:211-223.
26. Muller-Decker K, et al. The effects of cyclooxygenase isozyme inhibition on incisional wound healing in mouse skin. J Invest Dermatol 2002, 119:1189-1195.
27. Muscara M N, et al. Wound collagen deposition in rats: effects of an NO-NSAID and a selective COX-2 inhibitor. Br J Pharmacol 2000, 129:681-686.
28. Futagami A, et al. Wound healing involves induction of cyclooxygenase-2 expression in rat skin. Lab Invest 2002, 82:1503-1513.
29. Wilgus T A, et al. Reduction of scar formation in full-thickness wounds with topical celecoxib treatment. Wound Repair Regen 2003, 11:25-34.
30. Ashcroft G S, et al. Mice lacking Smad3 show accelerated wound healing and an impaired local inflammatory response. Nat Cell Biol 1999, 1:260-266.
31. Li-Korotky H S, et al. Identification of a pre-mRNA splicing factor, arginine/serine-rich 3 (Sfrs3), and its co-expression with fibronectin in fetal and postnatal rabbit airway mucosal and skin wounds. Biochim Biophys Acta. 2005 1762(1):34-45.
32. Dovi J V, et al. Accelerated wound closure in neutrophil-depleted mice. J Leukoc Biol 2003, 73:448-455.
33. Manus, M. T. and P. A. Sharp (2002) Gene silencing in mammals by small interfering RNAs. Nature Review, Genetics. 3(10):737-747.
34. Lu, P. Y. et al. (2003) siRNA-mediated antitumorigenesis for drug target validation and therapeutics. Current Opinion in Molecular Therapeutics. 5(3):225-234.
35. Kim, B. et al. (2004) Inhibition of ocular angiogenesis by siRNA targeting vascular endothelial growth factor-pathway genes; therapeutic strategy for herpetic stromal keratitis. Am. J. Pathol. 165 (6): 2177-85.
36. Tuschl, Zamore, Lehmann, Bartel and Sharp (1999), Genes & Dev. 13: 3191-3197.
37. Elbashir, Lendeckel and Tuschl (2001). Genes & Dev. 15: 188-200.
38. Lu, P. Y. and M. Woodle (2005) Delivering siRNA in vivo For functional genomics can novel therapeutics. In RNA Interference Technology. Cambridge University Press. P 303-317.
39. Lu, P. Y. et al. (2005) Modulation of angiogenesis with siRNA inhibitors for novel therapeutics. TRENDS in Molecular Medicine. 11(3), 104-13.
40. Lu P Y, Xie F, Woodle M C. (2005) In vivo application of RNA interference: from functional genomics to therapeutics. Adv Genet. 54:117-42.
41. Woodle, MC and PY Lu, Nanoparticles for RNAi Therapy. Nanotoday, August 2005, 34-41.
42. Xie, Y. F., M. Woodle and PY Lu. Harnessing in vivo siRNA delivery for functional genomics and novel therapeutics. Drug Discovery Today, January 2006.
43. Li B. J. et al, 2005, Nature Medicine, 11, 944-951.

1-46. (canceled)

47. A nucleic acid molecule that targets a sequence selected from the group consisting of:

| (SEQ ID NO: 11) | |
|---|---|
| a) | GAGUUAUGUGUUGACAUCCAGAUCA; |
| (SEQ ID NO: 12) | |
| b) | CAGAUCACAUUUGAUUGACAGUCCA; |
| (SEQ ID NO: 13) | |
| c) | GAAGCCUUCUCUAACCUCUCCUAUU; |
| (SEQ ID NO: 14) | |
| d) | CGACUCCCUUGGGUGUCAAAGGUAA; |
| (SEQ ID NO: 15) | |
| e) | CAGAUCAUAAGCGAGGGCCAGCUUU; |
| (SEQ ID NO: 16) | |
| f) | CAGUCAAAGAUACUCAGGCAGAGAU; |
| (SEQ ID NO: 17) | |
| g) | UCCAGACAAGCAGGCUAAUACUGAU; |
| (SEQ ID NO: 18) | |
| h) | CAACACUUGAGUGGCUAUCACUUCA; |
| (SEQ ID NO: 19) | |
| i) | GCAAACGCUUUAUGCUGAAGCCCUA; |
| | and |
| (SEQ ID NO: 20) | |
| j) | ACGCUUUAUGCUGAAGCCCUAUGAA. |

48. The nucleic acid molecule of claim 47, wherein the nucleic acid molecule is double-stranded and consists of a sequence selected from the group consisting of:

| (SEQ ID NO: 11) | |
|---|---|
| a) | GAGUUAUGUGUUGACAUCCAGAUCA; |
| (SEQ ID NO: 12) | |
| b) | CAGAUCACAUUUGAUUGACAGUCCA; |
| (SEQ ID NO: 13) | |
| c) | GAAGCCUUCUCUAACCUCUCCUAUU; |
| (SEQ ID NO: 14) | |
| d) | CGACUCCCUUGGGUGUCAAAGGUAA; |
| (SEQ ID NO: 15) | |
| e) | CAGAUCAUAAGCGAGGGCCAGCUUU; |
| (SEQ ID NO: 16) | |
| f) | CAGUCAAAGAUACUCAGGCAGAGAU; |
| (SEQ ID NO: 17) | |
| g) | UCCAGACAAGCAGGCUAAUACUGAU; |
| (SEQ ID NO: 18) | |
| h) | CAACACUUGAGUGGCUAUCACUUCA; |
| (SEQ ID NO: 19) | |
| i) | GCAAACGCUUUAUGCUGAAGCCCUA; |
| | and |
| (SEQ ID NO: 20) | |
| j) | ACGCUUUAUGCUGAAGCCCUAUGAA; |

and its complement.

49. The nucleic acid molecule of claim 47, comprising at least one nucleotide that is modified.

50. A composition comprising the nucleic acid molecule of claim 48 and a pharmaceutically acceptable carrier.

51. The composition of claim 50, further comprising one or more additional nucleic acid molecules that induce RNA interference and decrease the expression of a gene of interest.

52. The composition of claim 51, wherein the one or more additional nucleic acid molecules decrease the expression of a gene selected from the group consisting of TGF-β1 (Transforming Growth Factor beta 1), TGF-β2 (Transforming Growth Factor beta 2), interleukin-1, IL-6 (interleukin-6), IL-8 (interleukin-8), Hoxb13, Fibronectin, Smad3 (Transforming Growth Factor beta 1), Sfrs3 (Splicing factor, arginine/serine-rich 3), insulin-like growth factor I and platelet-derived growth factor.

53. The composition of claim 49, wherein the carrier is a nucleic acid delivery vehicle.

54. The composition of claim 53, wherein the nucleic acid delivery vehicle is synthetic.

55. The composition of claim 54 wherein the synthetic nucleic acid delivery vehicle comprises a cationic polymer-nucleic acid complex.

56. The composition of claim 55, wherein the cationic polymer is a histidine-lysine copolypeptide.

57. The composition of claim 56, wherein the synthetic nucleic acid delivery vehicle further comprises a hydrophilic polymer.

58. The composition of claim 57, wherein the hydrophilic polymer is polyethylene glycol (PEG).

59. The composition of claim 56, wherein the synthetic nucleic acid vehicle further comprises a targeting ligand.

60. The composition of claim 59, wherein the targeting ligand is a protein.

61. The composition of claim 59, wherein the targeting ligand binds an epithelial cell, a vascular endothelial cell, a vascular smooth muscle cell, a myocardial (heart) cell or a passenger leukocyte cell resident in cutaneous tissue at a time of wound healing.

62. The composition of claim 54, wherein the synthetic nucleic acid delivery vehicle comprises:
(a) a histidine-lysine co-polymer;
(b) a hydrophilic polymer comprising PEG; and, optionally,
(c) a targeting ligand.

63. The composition of claim 54, comprising an additional therapeutic agent that improves wound healing.

64. A method for decreasing the Cox-2 (Cyclooxygenase-2) protein level in a cell, comprising introducing into the cell the nucleic acid molecule of claim 48 or the composition of claim 50.

65. A method of reducing inflammation in a subject in need thereof, comprising the step of administering to the subject the nucleic acid molecule of claim 48 or the composition of claim 50.

66. A method of reducing scar formation in a subject in need thereof, comprising the step of administering to the subject the nucleic acid molecule of claim 48 or the composition of claim 50.
